(19) United States
(12) Patent Application Publication
(10) Pub. No.: US 2004/0156838 A1
(43) Pub. Date: Aug. 12, 2004

(54) Method for down-regulating ige

(76) Inventors: Steen Klysner, Horsholm (DK); Paul Von Hoegen, Hennigsdorf (DE); Bjorn Voldborg, Horsholm (DK); Anand Gautam, Niva (DK)

Correspondence Address: BIRCH STEWART KOLASCH & BIRCH, PO BOX 747, FALLS CHURCH, VA, 22040-0747, US

(21) Appl. No.: 10/363,954

(22) PCT Filed: Sep. 6, 2001
(86) PCT No.: PCT/DK01/00579

(30) Foreign Application Priority Data
Sep. 6, 2000 (DK) PA 2000 01326

(51) Int. Cl. A61K 48/00; A61K 39/395

(52) U.S. Cl. 424/130.1; 514/44

(57) ABSTRACT

The present invention discloses methods for immunizing against autologous (self) Immunoglobulin E (IgE). In particular, the invention discloses methods for inducing cytotoxic T-lymphocytes that will specifically down-regulate B-cells producing autologous IgE, notably by means of nucleic acid vaccination or live vaccination. Also disclosed are methods for inducing antibodies reactive with autologous IgE as well as methods for inducing a combined antibody and CTK response specific for IgE. The invention also discloses specific immunogenic protein constructs, nucleic acids encoding these as well as various formulations and tools for preparing the vaccines, including vectors and transformed host cells.

### FIELD OF THE INVENTION

The present invention relates to novel methods for combating allergy involving type I hypersensitivity. In particular, the present invention relates to methods for inducing an immune response conducted by cytotoxic T-lymphocytes (CTLs) against IgE producing B-cells, whereby these B-cells are attacked and killed by the CTLs.

### BACKGROUND OF THE INVENTION

Immunoglobulin E is the main effector in anaphylaxis and as such responsible for the initiation of a series of mechanisms which are triggered by the binding of an antigen to IgE on the surface of cells bearing the high affinity Fcε receptor (FcεRI).

While an anti-IgE response could be a useful rapid immune response against parasites, allergen induced IgE secretion can result in a variety of complications, including death, as may be the case in serious cases of asthma and anaphylaxis. These allergic disorders are prevalent. For example, allergic rhinitis (hay fever) affects 22% or more of the population of the USA, whereas allergic asthma is thought to affect at least 20 million residents of the USA. The economic impact of allergic diseases in the United States, including health care costs and lost productivity, was estimated to amount to $6.4 billion in early nineties alone. Moreover, the incidence of these IgE-associated disorders, at least in populations for which reliable data are available, appears to be increasing.

The role of increased IgE secretion in a majority of allergic diseases has been clearly established. Biological properties and how IgE may promote allergic symptoms are summarized below.

IgE not only has the shortest biologic half-life of all classes of immunoglobulins (Igs), but also is present in serum at the lowest levels. However, IgE concentrations in allergic reactions (atopic) in individuals can be 100- to 1000-fold higher than in normal individuals. IgE is directly involved in mediating many allergic reactions as a result of its ability to bind to and, upon contact with multivalent allergen, activate various effector cells, such as mast cells and basophils (see below)

The induction of IgE synthesis requires cytokines secreted by CD4+ T cells of T helper 2 (Th2) phenotypes. Th2 cells secrete IL-4, IL-5, IL-6, IL-10, and IL-13 that are important in the development of humeral immune responses, including IgE-associated allergic responses. T helper 1 (Th1) cells, on the other hand, secrete IL-2, γIFN and TNF, cytokines important in the development of cell mediated immune responses. These facts have supported the widely held view that undesired IgE-associated immune responses are the unfortunate outcome of the immune system perceiving and responding to otherwise essentially harmless allergens as if they were derived from parasites.

Mechanisms of IgE-Associated Acute, Late-Phase, and Chronic Allergic Diseases

Allergen challenge of sensitised individuals can elicit three types of responses: a) acute allergic reaction, b) late-phase reaction, and c) chronic allergic inflammation.

a) Acute allergic reaction: The major feature of the acute allergic reaction, which can be expressed seconds or minutes after exposure to allergen, primarily reflect the actions of mediators released from already IgE loaded mast cells and other effector cells that are normally resident in the tissue at the site of allergen challenge.

b) Late-phase allergic reaction: Some of the mediators that are released in response to acute allergic reaction, in addition to having direct effect on cells resident in the affected tissue, such as vascular endothelial cells, secretory glands, sensory nerves, and vascular, respiratory, or gastrointestinal smooth muscle cells, also have effects that result in recruitment of circulating leukocytes. Such recruited leukocytes can in turn influence the local characteristics of the evolving allergic responses, for example, by contributing to the reappearance or development of erythema (reflecting increased blood flow) and swelling (reflecting increased vascular permeability) in the skin or airway narrowing in the respiratory tract. These late-phase reactions characteristically do not develop until several hours after initial allergen challenge, in many cases after the signs and symptoms related to the acute allergic reaction have greatly diminished or even disappeared.

c) Chronic allergic inflammation: This typically occurs at anatomic sites that have been repeatedly challenged with allergen over prolonged periods. Sites of chronic allergic inflammation not only contain effector cells that have been recruited from the circulation, notable including increased number of eosinophils and T cells, many of them of the Th2 pheno-type, but can also be associated with striking chronic (i.e. long-lasting) changes in the underlying tissues. Human allergic asthma is a typical example of this where persistent insult by allergens can be associated with major structural changes in all layers of the affected airways. The repeated exposure to the allergen results in a marked elevation of total as well as allergen-specific IgE. This IgE in turns enhances the ability of mast cells and basophils to secrete IL-4, IL-13, and other mediators that can promote further IgE production. Secretion of these cytokines may also recruit and further activate Th2 cells for a cycle of Th2 cell-driven, IgE-associated immune responses.

Receptors for IgE Binding

The two major Fc receptors (Fcε) for IgE are distinguished by their structures and their relative affinities for IgE. The high affinity receptor for IgE (FcεRI), binds monomeric IgE with affinity (Kₐ) of about 10¹⁰ M⁻¹, while the second receptor for IgE, FcεRII (CD23), binds IgE with much lower affinity (Kₐ=10⁸ M⁻¹). A large proportion of studies have therefore been conducted on FcεR1.

Mast cells and basophils constitutively express high levels of FcεRI. Low levels of FcεRI can also be detected on human Langerhans' cells, peripheral dendritic cells, and monocytes, where it can function in IgE-mediated antigen presentation. In addition, FcεRI has been reported on eosinophils.

Many lines of evidence indicate that the activation of mast cells, basophils and even in some circumstances, eosinophils via FcεRI, resulting in the release of potent biologically active mediators, represents a primary (and in many cases, the primary) effector mechanism in allergic responses that are demonstrably IgE-dependent, such as those that can be transferred passively with antigen-specific IgE antibodies. The activation of mast cells and basophils by FcεRI aggregation initiates a coordinated sequence of biochemical and morphologic events that results in 1) exocytosis of secretory granules containing histamine and other preformed mediators; 2) synthesis and secretion of newly formed lipid mediators, such as prostaglandins and leukotrienes, 3) synthesis and secretion of Th2 cytokines (e.g. IL-4, IL-13, and MIP-1a) that can promote IgE production. Together, these mediators are responsible for the majority of the clinical symptoms associated with acute IgE-associated allergic reactions, and also contribute to the development of late phase reactions and chronic allergic inflammation. The crucial role of FcεRI has been demonstrated in mice with targeted gene disruption of the IgE-binding FcεRI a chain.

Studies in both mice and humans have revealed that levels of FcεRI surface expression on mast cells and basophils can be regulated by levels of IgE. Moreover, genetically IgE-deficient mice exhibit a dramatic (greater than 80%) reduction in mast cell and basophil FcεRI expression, which can be corrected by administration of monomeric IgE in vivo. While the mechanism(s) by which monomeric IgE regulates FcεRI expression is/are not yet clear, research in this area has already opened up novel therapeutic approaches for the management of allergic diseases.

Major IgE-Associated Human Diseases

1. Anaphylaxis

Anaphylaxis is an acute, systemic, hypersensitivity response to allergen, which typically involves multiple organ systems and which, if untreated, can rapidly lead to death. Such reaction can be elicited by allergens derived from diverse agents (e.g. venoms, airborne allergens, foods, antibiotics etc). It is widely believed that most, if not all, of the signs and symptoms are associated with an overproduction of IgE antibodies. This reflects 1) the systemic, FcεRI-dependent activation of mast cells and/or basophils and 2) the end-organ consequences of the release of mediators by these cells.

2. Allergic Rhinitis

As mentioned above, allergic rhinitis, commonly known as hay fever, inflicts about 22% of the population in the USA alone. Symptoms, which include sneezing, nasal congestion and itching, as well as rhinorrhea (increased production of nasal secretions), in most cases primarily reflect the IgE-dependent release of mediators by mast cells and basophils in response to airborne allergens. While, some of the pathophysiology of allergic rhinitis clearly reflects the consequence of locally elicited acute allergic reactions, a considerable amount of symptoms have a late phase reaction (delayed responses) and even chronic allergic inflammation due to massive recruitment of the effector cells and production of IgE and Th2 cytokines. Combination of these mediators, cytokines and cells perpetuates an IgE-dependent allergic disease process by mechanisms already discussed above.

3. Asthma

Asthma affects millions of people worldwide. The human and economic costs of this disorder (in morbidity, health care expenses, lost productivity, and most tragically, even mortality) are enormous. Rather than constituting a single “disease”, it is now generally thought that asthma is a syndrome typically characterized by three major features: 1) intermittent and reversible airway obstruction; 2) airway “hyperresponsiveness” (i.e., a markedly increased sensitivity of the airways, as reflected in bronchoconstriction, to immunologically non-specific stimuli such as histamine and cholinergic agonists); and 3) airway inflammation.

The syndrome of asthma may arise as a result of interaction between multiple genetic and environmental factors. Nevertheless, most cases of asthma disorder occur in subjects who also exhibit acute immediate hypersensitivity responses to defined environmental allergens. It is also known that the overall incidence of asthma exhibits a strong positive correlation with serum concentrations of IgE. Moreover, it has been shown that the high affinity IgE receptor, FcεRI, which was once though to be restricted to tissue mast cells and basophils, can also be expressed on the surface of monocytes, circulating dendritic cells, Langerhans' cells, and eosinophils, thus identifying these cells as additional potential sources of mediators in various IgE-dependent inflammatory responses.

Both eosinophils and Th2 cells are well represented in chronic inflammatory infiltrates in the airways of patients with asthma and can produce cytokines or other mediators that may contribute to many of the features of the disease. However, expression of FcεRI and serum levels of IgE switches the immune response mediated by the Th2 cytokines and recruitment of Th2 cells and eosinophils. Thus in humans, IgE may not only serve to arm mast cells and other effector cells, but may also contribute, by enhancing IgE production, to the further development of asthma syndrome.

4. Atopic Dermatitis

This prevalent and troublesome chronic skin disease can be regarded as the cutaneous manifestation of atopy (allergic reaction).

Anti-IgE Vaccination

It has previously been suggested to vaccinate against autologous IgE. EP-A-666760 suggests a vaccination strategy where a polypeptide conjugate including the CH2-CH3 domains (or parts thereof) of the IgE heavy chain are used as the immunogen. The rationale is to avoid cross-linking of FcεRI bound IgE on the surface of mast cells and basophils—since it is known that the FcεRI binding region is (partly) comprised of the so-called hinge-region between the CH2 and CH3 domains, the use of this region as the self-protein part of the immunogenic conjugate leads to induction of antibodies which ought to bind only soluble IgE.

A related approach was earlier suggested by Stanworth et al., which utilised short peptides from the CH4 domain conjugated to a carrier molecule.

Finally, a number of patent applications assigned to Tanox Biosystems (e.g. WO 89/06138) have focussed on passive immunization with antibodies reactive with the MIGIS fragment of B-cell bound IgE, i.e. the extracellular part of the membrane anchoring part of B-cell bound IgE. Also this short peptidic fragment is absent on FcεR-bearing cells, and therefore the passive immunization will exclusively target B-cell bound IgE. Tanox also suggests active vaccination in the form of immunization with anti-idiotypic antibodies against antibodies that react with either the MIGIS fragment or the receptor binding part of IgE.

Also WO 95/05849 suggests vaccination against IgE. This is done in the context of rendering IgE immunogenic by introducing one or more T helper epitopes by means of substitution in the IgE sequence while preserving a maximum number of B-cell epitopes of native IgE.

Induction of T-cell Help in General

Presentation of antigens has dogmatically been thought of as two discrete pathways, a class II exogenous and a class I endogenous pathway.

Briefly, a foreign protein from outside the cell or from the cell membrane is taken up by the APC as an endosome that fuses with an intracellular compartment containing proteolytic enzymes and MHC class II molecules. Some of the produced peptides bind to class II, which then are translocated to the cell membrane.

The class I endogenous pathway is characterised by the predominant presentation of cytosolic proteins. This is believed to occur by proteasome-mediated cleavage followed by transportation of the peptides into the endoplasmic reticulum (ER) via TAP molecules located in the membrane of the ER. In ER the peptides bind to class I followed by transportation to the plasma membrane.

However, these 2 pathways are not fully distinct. For example it is known that dendritic cells and to some extend macrophages are capable of endocytosing (pinocytosing) extracellular proteins and subsequently present them in the context of MHC class I. It has also previously been demonstrated that using specialised administration routes, e.g. by coupling to iron oxide beads, exogenous antigens are capable of entering the Class I pathway (Rock, 1996). This mechanism seems central, because of the importance of a concomitant expression of both class I and class II on the same APC to elicit a three cell type cluster. This three-cell type cluster of interaction has been proposed by Mitchison (1987) and later by other authors. They showed the importance of concomitant presentation of class I and class II epitopes on the same APC. According to the recently described mechanism for CTL activation (cf. Lanzavecchia, 1998, Nature 393: 413, Matzinger, 1999, Nature Med. 5: 616, Ridge et al., 1998, Nature 393: 474, Bennett et al., 1998, Nature 393: 478, Schoenberger et al., 1998, Nature 393: 480, Ossendrop et al., 1998, J. Exp. Med 187: 693, and Mackey et al., 1998, J. Immunol 161: 2094), professional APCs presenting antigen on MHC class II are recognized by T helper cells. This results in an activation of the APC (mediated by interaction by CD40L on the T helper cell and CD40 on the APC). This enables the APC to directly stimulate CTLs that are thereby activated. Cf. also FIG. 2.

It has previously been demonstrated that insertion of a foreign MHC class II restricted T helper cell epitope into a self-antigen results in the provision of an antigen capable of inducing strong cross-reactive antibody responses directed against the non-modified self-antigen (cf. WO 95/05849). It was shown that the autoantibody induction is caused by specific T cell help induced by the inserted foreign epitope.

Later, it was concluded that modified self-antigens—with the aid of appropriate adjuvants—ought to be capable of also inducing strong CTL responses against MHC class I restricted self-epitopes and hence the technology described in WO 95/05849 can be adapted to also provide vaccination against intracellular and other cell-associated antigens which have epitopes presented in the context of MHC Class I—this concept is the subject matter of WO 00/20027 which is hereby incorporated by reference herein.

To the best of the present inventors knownledge, it has never been suggested to use nucleic acid vaccination against IgE. Neither has it been suggested to vaccinate so as to induce cytotoxic lymphocytes reactive with IgE producing B cells.

### OBJECT OF THE INVENTION

It is an object of the present invention to provide improved methods and agents for inducing immune responses in host organisms against IgE. It is a further object to provide a method for preparing polypeptide analogues of IgE, analogues that are capable of inducing an effective immune response against IgE.

### SUMMARY OF THE INVENTION

The present invention is in part based on a thorough analysis of the possible ways of reducing type I hypersensitivity via immunological modulation of IgE abundance.

In one aspect of the invention it has been concluded that induction of CTL responses against IgE producing B-cells will be an effective means for reducing IgE abundance. Since IgE producing B-cells do not seem to be of crucial importance for humans, it would therefore be relevant to reduce the number of IgE producing cells in circulation, thereby reducing the abundance of IgE.

In another aspect of the invention it has been concluded that DNA vaccination (apart from its ability to invoke CTLs) will be an effective means of immunizing against IgE, i.a. because it is possible to force through a shift from Th2 to Th1 cells—this is a consequence of the inherent quality of DNA vaccination to be capable of preferentially induce Th1 help.

In a third, and broad, aspect it has been concluded that, even though previous work with administration of monoclonal anti-IgE has demonstrated that cross-linking of FcεR-bound IgE and associated degranulation of mast cells and basophils occur, it is not altogether clear that immunization with an agent which gives rise to a broad-spectred polyclonal-antibody response against IgE will have the same undesired effects. Or, in other words, it is believed that induction of a polyclonal anti-IgE response will be effective in reducing IgE without suffering the drawback of stimulating degranulation.

We have in part based the present invention on the teachings of WO 00/20027—it has now been realized that induction of a CTL response against IgE producing cells will provide a beneficial down-regulation of B-cells producing IgE. This will, in turn, lead to a lowering of the level of both circulating as well as receptor-bound IgE.

Using the autovaccine constructs and vaccination protocol described in WO 00/20027, the modified IgE could be presented by MHC class I as well as by MHC class II molecules on professional antigen presenting cells. Co-presentation of subdominant self-epitopes on MHC class I and immunodominant foreign epitopes on MHC class II molecules would mediate a direct cytokine help from activated MHC class II restricted T-helper cells to MHC class I restricted CTLs (FIG. 2). This will lead to a specific break of the T cell autotolerance towards IgE.

In conclusion, a vaccine constructed using both of the technologies outlined above will induce a humeral autoantibody response with secondary activation of complement and antibody dependent cellular cytotoxicity (ADCC) activity. More important, it will also induce a cytotoxic T cell response directed against autologous IgE producing cells.

Hence, in the broadest and most general scope, the present invention relates to a method for inducing an immune response against autologous immunoglobulin E (IgE) in an animal, including a human being, the method comprising effecting simultaneous presentation by antigen presenting cells (APCs) of the animal's immune system of an immunogenically effective amount of

at least one CTL epitope derived from the autologous IgE and/or at least one B-cell epitope derived from the autologous IgE, and

at least one first T helper cell epitope (T_{H} epitope) which is foreign to the animal.

In a more specific variant of the inventive method, the invention relates to a method for down-regulating autologous IgE in an animal, including a human being by inducing a specific cytotoxic T-lymphocyte (CTL) response against cells producing autologous IgE, the method comprising effecting, in the animal, simultaneous presentation by a suitable antigen presenting cell (APC) of

at least one CTL epitope derived from IgE of the animal, and

at least one first T-helper lymphocyte (TH) epitope which is foreign to the animal.

Also, the novel strategy for preparing an immunogenic agent is part of the invention. This novel strategy encompasses the selection and production of analogues of IgE; where the preservation of a substantial fraction of known and predicted CTL epitopes is aimed at while at the same time introducing at least one foreign T_{H} epitope.

Furthermore, the invention relates to certain specific immunogenic constructs based on mammalian IgE as well as to compositions containing these constructs.

Finally, the invention relates to nucleic acid fragments, vectors, transformed cells and other tools useful in molecular biological methods for the production of the analogues of IgE.

### LEGENDS TO THE FIGURE

FIG. 1: The traditional AutoVac concept. A: Tolerodominant self-epitopes presented on MHC class II on an antigen presenting cell (APC) are ignored due to depletion in the T helper cell (Th) repertoire (T helper cell indicated with dotted lines). Inserted foreign immunodominant T cell epitopes presented on MHC class II activate T helper cells and B cells (B) specific for native parts of the self-protein presenting foreign immunodominant T cell epitopes on MHC class II are activated by the cytokine help provided by the T helper cell.

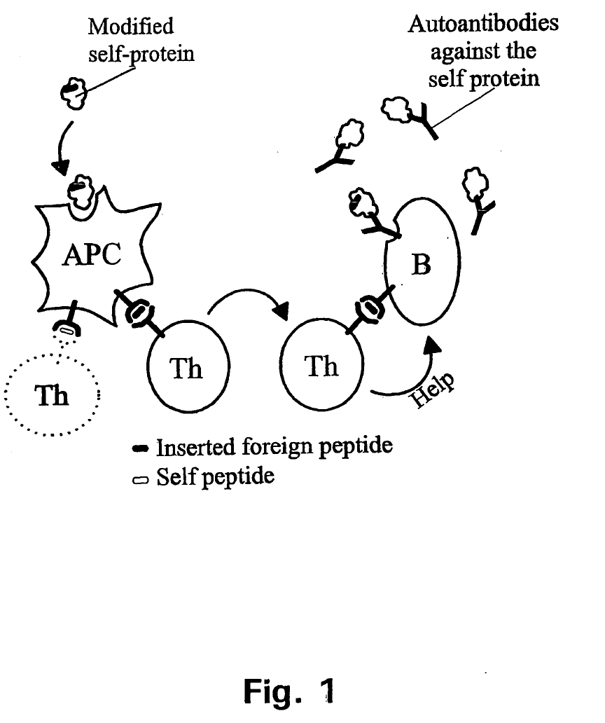

FIG. 2: The AutoVac concept for inducing a CTL response as disclosed in WO 00/20027. Inserted foreign immunodominant T cell epitopes presented on MHC class II activate T helper cells. CTLs recognising subdominant self-epitopes presented on MHC class I are activated by the adjacent activated T helper cell.

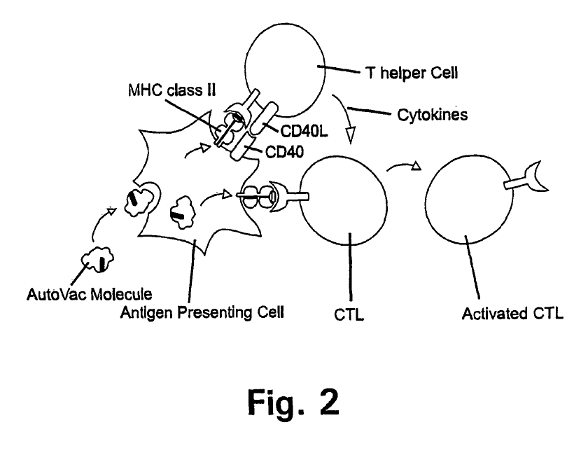

FIG. 3: Schematic representation of some preferred IgE-based immunogenic constructs.

Constructs based solely on the CH3 domain (C3) can include the P2 and P30 epitopes from tetanus toxoid in the form of additions (N— or C-terminal), insertions or substitutions. It is also a possibility to include the amino acid sequence of the MIGIS fragment in a similar manner. Constructs based on the CH3 (C3) and CH4 (C4) domains can include the P2 and/or P30 epitopes and the MIGIS fragments in a similar manner but also as an insertion or substitution between the two domains. The dark grey area in the CH3 domain indicates the FcεRI binding region.

### DETAILED DISCLOSURE OF THE INVENTION

Definitions

In the following a number of terms used in the present specification and claims will be defined and explained in detail in order to clarify the metes and bounds of the invention.

An “autologous IgE” is in the present specification and claims intended to denote an IgE polypeptide of an animal which is going to be vaccinated against its own IgE. It is understood that the term generally relates the non-variable parts of IgE (i.e. to the constant parts of the heavy or light chains), meaning that the various isoforms of the constant domains of IgE are encompassed by the term, whereas the variable domains are not regarded as being part of autologous IgE.

The terms “T-lymphocyte” and “T-cell” will be used interchangeably for lymphocytes of thymic origin which are responsible for various cell mediated immune responses as well as for effector functions such as helper activity in the humeral immune response. Likewise, the terms “B-lymphocyte” and “B-cell” will be used interchangeably for antibody-producing lymphocytes.

An “antigen presenting cell” (APC) is a cell which presents epitopes to T-cells. Typical antigen-presenting cells are macrophages, dendritic cells and other phagocytizing and pinocytizing cells. It should be noted that B-cells also functions as APCs by presenting T_{H} epitopes bound to MCH class II molecules to T_{H} cells but when generally using the term APC in the present specification and claims it is intended to refer to the above-mentioned phagocytizing and pinocytizing cells.

“Helper T-lymphocytes” or “T_{H} cells” denotes CD4 positive T-cells which provide help to B-cells and cytotoxic T-cells via the recognition of T_{H} epitopes bound to MHC Class II molecules on antigen presenting cells.

The term “cytotoxic T-lymphocyte” (CTL) will be used for CD8 positive T-cells which require the assistance of T_{H} cells in order to become activated.

A “specific” immune response is in the present context intended to denote a polyclonal immune response directed predominantly against a molecule or a group of quasi-identical molecules or, alternatively, against cells which present CTL epitopes of the molecule or the group of quasi-identical molecules.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same IgE allowing for immunization of the animals with the same immunogen(s). If, for instance, genetic variants of IgE exist in different human populations it may be necessary to use different immunogens in these different populations in order to be able to break the autotolerance towards the IgE in each population.

By the term “down-regulation a autologous IgE” is herein meant reduction in the living organism of the amount and/or activity of IgE. The down-regulation can be obtained by means of several mechanisms, including interference with the Fc&R binding region, removal of the IgE by scavenger cells (such as macrophages and other phagocytizing cells), and even more important, that cells carrying or harbouring the antigen are killed by CTLs in the animal.

The expression “effecting simultaneous presentation by a suitable APC” is intended to denote that the animal's immune system is subjected to an immunogenic challenge in a controlled manner which results in the simultaneous presentation by APCs of the IgE epitopes and foreign epitopes in question. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with APCs displaying the relevant epitopes in an immunologically effective manner.

The term “immunogen” is intended to denote a substance which is capable of inducing an immune response in a certain animal. It will therefore be understood that autologous IgE is not an immunogen in the autologous host—it is necessary to use either a strong adjuvant and/or to co-present T helper epitopes with the autologous IgE in order to mount an immune response against autologous IgE and in such a case the “immunogen” is the composition of matter which is capable of breaking autotolerance.

The term “immunogenically effective amount” has its usual meaning in the art, i.e. an amount of an immunogen which is capable of inducing an immune response which significantly engages pathogenic agents which share immunological features with the immunogen.

When using the expression that the autologous IgE has been subjected to a “modification” is herein meant a chemical modification of the polypeptide which constitutes at least part of one of the constant domains of autologous IgE. Such a modification can e.g. be derivatization (e.g. alkylation) of certain amino acid residues in the amino acid sequence, but as will be appreciated from the disclosure below, the preferred modifications comprise changes of the primary structure of the amino acid sequence.

When discussing “tolerance” and “autotolerance” is understood that since IgE molecules which are the targets of the present inventive method are self-proteins in the population to be vaccinated, normal individuals in the population do not mount an immune response against IgE. It cannot be excluded, though, that occasional individuals in an animal population might be able to produce antibodies against the autologous IgE, e.g. as part of a autoimmune disorder. At any rate, an animal will normally only be autotolerant towards its own IgE, but it cannot be excluded that analogues derived from other animal species or from a population having a different phenotype would also be tolerated by said animal.

A “foreign T-cell epitope” is a peptide which is able to bind to an MHC molecule and stimulates T-cells in an animal species. Preferred foreign epitopes are “promiscuous” epitopes, i.e. epitopes which binds to a substantial fraction of MHC class II molecules in an animal species or population. A term which is often used interchangeably in the art is the term “universal T-cell epitopes” for this kind of epitopes. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same analogue or 2) prepare several analogues wherein each analogue has a different promiscuous epitope inserted. It should be noted that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule. It is also important to add that the “foreignness” feature therefore has two aspects: A foreign T_{H} epitope is 1) presented in the MHC Class II context by the animal in question and 2) the foreign epitope is not derived from the same polypeptide as the target antigen for the immunization.

A “CTL epitope” is a peptide which is able to bind to an MHC class I molecule.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. For instance it is possible to use certain cytokines as a modifying moiety in the analogue (cf. the detailed discussion below), and in such a case, the issue of stability may be irrelevant since the coupling to the analogue provides the stability necessary.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen

### PREFERRED EMBODIMENTS

In essence, induction of active immunity against IgE may target IgE in 3 different locations: 1) Bound to the surface of B-cells, 2) in soluble form and 3) bound to the Fc& receptor on effector cells such as mast cells and basophils. A vaccine construct which can accomplish all 3 goals without inducing undesirable side effects in the form of degranulation of FcεR bearing cells would be a superior medicament in the treatment and prophylaxis of IgE mediated pathologies.

In order to induce a CTL response against a cell which presents epitopes derived from the autologous IgE on its surface, it is normally necessary that at least one CTL epitope, when presented, is associated with an MHC Class I molecule on the surface of the APC. Furthermore it is preferred that the at least one first foreign T_{H} epitope, when presented, is associated with an MHC Class II molecule on the surface of the APC.

Preferred APCs presenting the epitopes are dendritic cells and macrophages, but any pino- or phagocytizing APC which is capable of simultaneously presenting 1) CTL epitopes bound to MHC class I molecules and 2) T_{H} epitopes bound to MHC class II molecules, is a preferred APC according to the invention.

Normally, it will be advantageous to confront the immune system with a large fraction of the amino acid sequence of the autologous target IgE. Hence, in a preferred embodiment, presentation by the APC of the CTL epitope and the first foreign T_{H} epitope is effected by presenting the animal's immune system with at least one first analogue of the autologous IgE, said first analogue comprising a variation of the amino acid sequence of the autologous IgE, said variation containing at least the CTL epitope and the first foreign T_{H} epitope. This is in contrast to e.g. a DNA vaccination strategy where the CTL and T_{H} epitopes are expressed by the same cell but as parts of separate polypeptides; such a DNA vaccination strategy is also an embodiment of the invention, but it is believed that having the two epitopes as part of the same polypeptide will normally enhance the immune response and, at any rate, the provision of only one expression product will be necessary.

In order to maximize the chances of mounting an effective immune response, it is preferred that the above-mentioned first analogue contains a substantial fraction of known and predicted CTL epitopes of autologous IgE, i.e. a fraction of the known and predicted CTL epitopes which binds a sufficient fraction of MHC Class I molecules in a population. For instance, it is preferred that the substantial fraction of known and predicted CTL epitopes in the amino acid sequence of the analogue are recognized by at least 50% of the MHC-I haplotypes recognizing all known and predicted CTL epitopes in the autologous IgE, but higher percentages are preferred, such as at least 60, at least 70, at least 80, and at least 90%. Especially preferred is the use of analogues which preserve substantially all known CTL epitopes of the autologous IgE are present in the analogue, i.e. close to 100% of the known CTL epitopes. Accordingly, it is also especially preferred that substantially all predicted CTL epitopes of the autologous IgE are present in the at least first analogue.

The above-indicated approach renders possible the mounting of a CTL response against all parts of B-cell associated IgE, including the transmembrane and intracellular parts of the membrane-anchoring region.

Methods for predicting the presence of CTL epitopes are well-known in the art, cf. e.g. Rothbard et al. EMBO J. 7:93-100 (1988).

As will be apparent from the present specification and claims it is expected that the inventive method described herein will render possible the effective induction of CTL responses against autologous IgE.

Since IgE in B-cells is a membrane-associated antigen, it is advantageous to induce an antibody response while at the same time inducing CTL mediated immunity. However, when raising a humeral immune response against autologous IgE it is preferred to substantially restrict the antibody response to interaction with the parts of the antigen which are normally exposed to possible interaction with antibodies. Otherwise the result would most likely be the induction of an antibody response against parts of the antigen which is not normally engaging the humeral immune system (e.g. the transmembrane and intracellular parts of the membrane anchoring region of B-cell bound IgE), and this will in turn increase the risk of inducing cross-reactivity with antigens not related to any pathology. One elegant way of obtaining this restriction is to perform nucleic acid vaccination with an analogue of autologous IgE, where the extracellular part thereof is either unaltered or includes a T_{H} epitope which does not substantially alter the 3D structure of the relevant extracellular part of the antigen. As one possible alternative, immunization can be performed with both a CTL directed immunogen and a B-cell directed immunogen where the B-cell directed immunogen is substantially incapable of effecting immunization against the intracellular part of the target antigen (the B-cell directed immunogen could e.g. lack any non-extracellular material from the antigen).

Induction of antibody responses can be achieved in a number of ways known to the person skilled in the art. For instance, the at least one first analogue may comprise a part consisting of a modification of the structure of the autologous IgE, said modification having as a result that immunization of the animal with the first analogue also induces production of antibodies in the animal against the autologous IgE—this variant is as mentioned above especially suited for nucleic acid vaccination. Alternatively, the method of the invention can involve effecting presentation to the animal's immune system of an immunogenically effective amount of at least one second analogue of the autologous IgE which contains such a modification. A convenient way to achieve that the modification has the desired antibody-inducing effect is to include at least one second foreign T_{H} epitope in the second analogue, i.e. a strategy like the one used for the first analogue.

In the cases where it is desired to also mount an effective humeral immune response, it is advantageous that the first and/or second analogue(s) comprise(s) a substantial fraction of the B-cell epitopes of autologous IgE, especially a substantial fraction of such B-cell epitopes which are extracellular in the naturally occurring form of autologous IgE.

The above-discussed variations and modifications of the autologous IgE can take different forms. It is preferred that the variation and/or modification involves amino acid substitution and/or deletion and/or insertion and/or addition. These fundamental operations relating to the manipulation of an amino acid sequence are intended to cover both single-amino acid changes as well as operations involving stretches of amino acids (i.a. shuffling of amino acid stretches within the polypeptide antigen; this is especially interesting when the antigenic determinant is from the intracellular part of B-cell associated IgE, since only considerations concerning preservation of CTL epitopes are relevant). It will be understood, that the introduction of e.g. one single amino acid insertion or deletion may give rise to the emergence of a foreign T_{H} epitope in the sequence of the analogue, i.e. the emergence of an MHC Class II molecule binding sequence. However, in most situations it is preferable (and even necessary) to introduce a known foreign T_{H} epitope, and such an operation will require amino acid substitution and/or insertion (or sometimes addition in the form of either conjugation to a carrier protein or provision of a fusion polypeptide by means of molecular biology methods. It is preferred that the number of amino acid insertions, deletions, substitutions or additions is at least 2, such as 3, 4, 5, 6, 7, 8, 9, 10, 11, 12, 13, 14, 15, 16, 17, 18, 19, 20, and 25 insertions, substitutions, additions or deletions. It is furthermore preferred that the number of amino acid substitutions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 30.

Preferred embodiments of the invention include modification by introducing at least one foreign immunodominant T_{H} epitope. It will be understood that the question of immune dominance of a T-cell epitope depends on the animal species in question. As used herein, the term “immunodominance” simply refers to epitopes which in the vaccinated individual/population gives rise to a significant immune response, but it is a well-known fact that a T-cell epitope which is immunodominant in one individual is not necessarily immunodominant in another individual of the same species, even though it may be capable of binding MHC-II molecules in the latter individual. True immune dominant T_{H} epitopes are those which, independent of the polypeptide wherein they form a subsequence, give rise to activation of T_{H} cells—in other words, some T_{H} epitopes have, as an intrinsic feature, the characteristic of substantially never being cryptic since they are substantially always processed by APCs and presented in the context of an MHC II molecule on the surface of the APC.

Another important point is the issue of MHC restriction of T-cell epitopes. In general, naturally occurring T-cell epitopes are MHC restricted, i.e. a certain peptides constituting a T-cell epitope will only bind effectively to a subset of MHC Class II molecules. This in turn has the effect that in most cases the use of one specific T-cell epitope will result in a vaccine component which is only effective in a fraction of the population, and depending on the size of that fraction, it can be necessary to include more T-cell epitopes in the same molecule, or alternatively prepare a multi-component vaccine wherein the components are variants of the antigen which are distinguished from each other by the nature of the T-cell epitope introduced.

If the MHC restriction of the T-cells used is completely unknown (for instance in a situation where the vaccinated animal has a poorly defined MHC composition), the fraction of the animal population covered by a specific vaccine composition can be determined by means of the following formula:

$${f}_{population} = - {\prod }_{i =}^{n} - {p}_{i} \qquad ( II )$$

where pᵢ is the frequency in the population of responders to the i^{th} foreign T-cell epitope present in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give

1−0.2×0.3×0.4=0.976

i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = - {\prod }_{j =} - {ϕ}_{j} \qquad ( III )$$

wherein φⱼ is the sum of frequencies in the population of allelic haplotypes encoding MHC molecules which bind any one of the T-cell epitopes in the vaccine and which belong to the j^{th} of the 3 known HLA loci (DP, DR and DQ); in practice, it is first determined which MHC molecules will recognize each T-cell epitope in the vaccine and thereafter these MHC molecules are listed by type (DP, DR and DQ)—then, the individual frequencies of the different listed allelic haplotypes are summed for each type, thereby yielding φ₁, φ₂, and φ₃.

It may occur that the value pi in formula II exceeds the corresponding theoretical value πᵢ:

$${π}_{i} = - {\prod }_{j =} - {v}_{j} \qquad ( IV )$$

wherein vⱼ is the sum of frequencies in the population of allelic haplotypes encoding MHC molecules which bind the i^{th} T-cell epitope in the vaccine and which belong to the j^{th} of the 3 known HLA loci (DP, DR and DQ). This means that in 1−πᵢ of the population there is a frequency of responders of f_{residual}_{_{—}}ᵢ=(pᵢ−πᵢ)/(1−πᵢ). Therefore, formula III can be adjusted so as to yield formula V:

$${f}_{population} = - {\prod }_{j =} - {ϕ}_{j} + ( - {\prod }_{i =}^{n} - {f}_{residual\_i} ) \qquad ( V )$$

where the term 1−f_{residual}_{_{—}}ᵢ is set to zero if negative. It should be noted that formula V requires that all epitopes have been haplotype mapped against identical sets of haplotypes.

Therefore, when selecting T-cell epitopes to be introduced in the analogue, it is important to include all knowledge of the epitopes which is available: 1) The frequency of responders in the population to each epitope, 2) MHC restriction data, and 3) frequency in the population of the relevant haplotypes.

There exist a number of naturally occurring “promiscuous” T-cell epitopes which are active in a large proportion of individuals of an animal species or an animal population and these are preferably introduced in the vaccine thereby reducing the need for a very large number of different analogues in the same vaccine.

The promiscuous epitope can according to the invention be a naturally occurring human T-cell epitope such as epitopes from tetanus toxoid (e.g. the P2 and P30 epitopes, cf. SEQ ID NOs: 12 and 14 in WO 00/20027), diphtheria toxoid, Influenza virus hemagluttinin (HA), and P. falciparum CS antigen.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in analogues used according to the present invention. Cf. also the epitopes discussed in the following references which are hereby all incorporated by reference herein: WO 98/23635 (Frazer I H et al., assigned to The University of Queensland); Southwood S et. al, 1998, J. Immunol. 160: 3363-3373; Sinigaglia F et al., 1988, Nature 336: 778-780; Rammensee H G et al., 1995, Immunogenetics 41: 4 178-228; Chicz R M et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes which share common motifs with these.

Alternatively, the epitope can be any artificial T-cell epitope which is capable of binding a large proportion of haplotypes. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the modified IgE which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA (SEQ ID NO: 18) or an immunologically effective subsequence thereof. This, and other epitopes having the same lack of MHC restriction are preferred T-cell epitopes which should be present in the analogues used in the inventive method. Such super-promiscuous epitopes will allow for the most simple embodiments of the invention wherein only one single analogue is presented to the vaccinated animal's immune system.

The nature of the above-discussed variation/modification preferably comprises that

at least one first moiety is included in the first and/or second analogue(s), said first moiety effecting targeting of the analogue to an antigen presenting cell (APC), and/or

at least one second moiety is included in the first and/or second analogue(s), said second moiety stimulating the immune system, and/or

at least one third moiety is included in the first and/or second analogue(s), said third moiety optimising presentation of the analogue to the immune system.

The functional and structural features relating these first, second and third moieties will be discussed in the following:

They can be present in the form of side groups attached covalently or non-covalently to suitable chemical groups in the amino acid sequence of the autologous IgE or a subsequence thereof. This is to mean that stretches of amino acid residues derived from the autologous IgE are derivatized without altering the primary amino acid sequence, or at least without introducing changes in the peptide bonds between the individual amino acids in the chain.

The moieties can also be in the form of fusion partners to the amino acid sequence derived from the autologous IgE. In this connection it should be mentioned that both possibilities include the option of conjugating the amino acid sequence to a carrier, cf. the discussion of these below. In other words, in the present context the term “fusion protein is not merely restricted to a fusion construct prepared by means of expression of a DNA fragment encoding the construct but also to a conjugate between two proteins which are joined by means of a peptide bond in a subsequent chemical reaction.

As mentioned above, the analogue can also include the introduction of a first moiety which targets the analogue to an APC or a B-lymphocyte. For instance, the first moiety can be a specific binding partner for a B-lymphocyte specific surface antigen or for an APC specific surface antigen. Many such specific surface antigens are known in the art. For instance, the moiety can be a carbohydrate for which there is a receptor on the B-lymphocyte or the APC (e.g. mannan or mannose). Alternatively, the second moiety can be a hapten. Also an antibody fragment which specifically recognizes a surface molecule on APCs or lymphocytes can be used as a first moiety (the surface molecule can e.g. be an FCγ receptor of macrophages and monocytes, such as FCγRI or, alternatively any other specific surface marker such as CD40 or CTLA-4). It should be noted that all these exemplary targeting molecules can be used as part of an adjuvant, cf. below. CD40 ligand, antibodies against CD40, or variants thereof which bind CD40 will target the analogue to dendritic cells. At the same time, recent results have shown that the interaction with the CD40 molecule renders the T_{H} cells unessential for obtaining a CTL response. Hence, it is contemplated that the general use of CD40 binding molecules as the first moiety (or as adjuvants, cf. below) will enhance the CTL response considerably; in fact, the use of such CD40 binding molecules as adjuvants and “first moieties” in the meaning of the present invention is believed to be inventive in its own right.

As an alternative or supplement to targeting the analogue to a certain cell type in order to achieve an enhanced immune response, it is possible to increase the level of responsiveness of the immune system by including the above-mentioned second moiety which stimulates the immune system. Typical examples of such second moieties are cytokines, heat-shock proteins, and hormones, as well as effective parts thereof.

Suitable cytokines to be used according to the invention are those which will normally also function as adjuvants in a vaccine composition, e.g. interferon γ (IFN-γ), Flt3 ligand (Flt3L), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF); alternatively, the functional part of the cytokine molecule may suffice as the second moiety. With respect to the use of such cytokines as adjuvant substances, cf. the discussion below.

Alternatively, the second moiety can be a toxin, such as listeriolycin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities.

According to the invention, suitable heat shock proteins used as the second moiety can be HSP70, HSP90, HSC70, GRP94, and calreticulin (CRT).

Also the possibility of introducing a third moiety that enhances the presentation of the analogue to the immune system is an important embodiment of the invention. The art has shown several examples of this principle. For instance, it is known that the palmitoyl lipidation anchor in the Borrelia burgdorferi protein OspA can be utilised so as to provide self-adjuvating polypeptides (cf. e.g. WO 96/40718). It seems that the lipidated proteins form up micelle-like structures with a core consisting of the lipidation anchor parts of the polypeptides and the remaining parts of the molecule protruding therefrom, resulting in multiple presentations of the antigenic determinants. Hence, the use of this and related approaches using different lipidation anchors (e.g. a myristyl group, a farnesyl group, a geranyl-geranyl group, a GPI-anchor, and an N-acyl diglyceride group) are preferred embodiments of the invention, especially since the provision of such a lipidation anchor in a recombinantly produced protein is fairly straight-forward and merely requires use of e.g. a naturally occurring signal sequence as a fusion partner for the analogue. Another possibility is use of the C3d fragment of complement factor C3 or C3 itself (cf. Dempsey et al., 1996, Science 271, 348-350 and Lou & Kohler, 1998, Nature Biotechnology 16, 458-462).

It is important to note that when attempting to use the method of the invention against epitopes of the extracellularly exposed parts of IgE, it is most preferred that the first and/or second analogue(s) has/have substantially the overall tertiary structure of one or more constant domains of IgE heavy or light chain. Thus, in the present specification and claims this is intended to mean that the overall tertiary structure of the part of IgE which is extracellularly exposed is preserved, since, as mentioned above, the tertiary structure of the obligate intracellular part (such as the intracellular part of the B-cell membrane anchoring region) do not engage the humeral immune system. In fact, as part of the vaccination strategy of the present invention it is often desired to avoid exposure to the extracellular compartment of putative B-cell epitopes derived from intracellular part of IgE; in this way, potentially adverse effects caused by cross-reactivity with other antigens can be minimized.

For the purposes of the present invention, it is however sufficient if the variation/modification (be it an insertion, addition, deletion or substitution,) gives rise to a foreign T-cell epitope and at the same time preserves a substantial number of the CTL epitopes in IgE (and sometimes also a substantial number of B-cell epitopes).

The following formula describes the constructs generally covered by the invention:

(MOD₁)ₛ₁(IgEₑ₁)ₙ₁(MOD₂)ₛ₂(IgEₑ₂)ₙ₂ . . . (MODₓ)ₛₓ(IgEₑₓ)ₙₓ (I)

where IgEₑ₁−IgEₑₓ are x CTL and/or B-Cell epitope containing subsequences of the autologous IgE which independently are identical or non-identical and which may contain or not contain foreign side groups, x is an integer ≧3, n1−nx are x integers ≧0 (at least one is ≧1), MOD₁−MODₓ are x modifications introduced between the preserved epitopes, and s₁−sx are x integers ≧0 (at least one is ≧1 if no side groups are introduced in the sequences). Thus, given the general functional restraints on the immunogenicity of the constructs, the invention allows for all kinds of permutations of the original constant IgE heavy or light chain sequence, and all kinds of modifications therein. Thus, included in the invention are analogues obtained by omission of parts of the autologous IgE sequence which e.g. exhibit adverse effects in vivo (such as parts of the CH1 domain of the heavy chain of IgE or omission of parts which are normally intracellular and thus could give rise to undesired immunological reactions, cf. the detailed discussion below.

If it should come out that there are serious adverse effects involved when immunizing with immunogens capable of raising antibodies against large parts of autologous IgE, it is preferred to restrict the antibody response to be directed against “safe” regions of IgE. For example, since it has previously been demonstrated that immunization with the complete CH2-CH3 domains (and also the CH3 domain alone) does not lead to degranulation of mast cells due to cross-linking of FcεR bound IgE, it therefore is logical to include B-cell epitopes derived from these domains—notably, the hinge region between the CH2 and CH3 domains is known to include the 76 amino acids FcεRI binding part of IgE, and use of this specific region will ensure that no cross-linking can take place. Further, the extracellular part of the membrane anchoring region of B-cell bound IgE (the MIGIS fragment) also include interesting epitopes which will not be capable of inducing cross-linking antibodies. Finally, recent research has revealed that the CH4 domain of IgE is also involved in the events leading to binding to FcεRI binding of IgE. Therefore, the immunogen used in the present invention preferably includes at least one B-cell epitope from the CH2 domain and/or from the CH3 domain and/or from the CH4 domain and/or from the MIGIS fragment of the autologous IgE. In preferred embodiments, the immunogen (e.g. the first and/or second analogues) include the complete CH3 and CH4 domains where at least one foreign T helper epitope is introduced by means of insertion or substitution. Such a construct can also include the MIGIS fragment.

Especially preferred constructs of the present invention include or consist of the structure:

I₁—(CH3)ₙ₁-I₂—(CH4)ₙ₂-I₃

where CH3 is the complete CH3 domain of autologous IgE, CH4 is the complete CH4 domain of autologous IgE, and I₁, I₂ and I₃ are amino acid sequences which each incorporates at least one foreign T helper epitope and/or the MIGIS fragment of autologous IgE, and n1 and n2 are integers ≧0, where at least one is ≧1. Alternatively, the constructs of the present invention include the foreign T-cell epitope as a substituent in the CH3 or CH4 domains, while at the same time ensuring that the tertiary structure of the domain of choice is not affected significantly by the substitution.

It is furthermore preferred that the variation and/or modification includes duplication, when applicable, of the at least one B-cell epitope, or of at least one CTL epitope of the autologous IgE. This strategy will give the result that multiple copies of preferred epitopic regions are presented to the immune system and thus maximizing the probability of an effective immune response. Hence, this embodiment of the invention utilises multiple presentations of epitopes derived from the autologous IgE (i.e. formula I wherein at least one B-cell epitope is present in two positions).

This effect can be achieved in various ways, e.g. by simply preparing fusion polypeptides comprising the structure (IgEₑ)ₘ, where m is an integer ≧2 and IgEₑ is a region of constant IgE heavy or light chain containing at least one CTL or B-cell epitope and then introduce the modifications discussed herein in at least one of the epitope containing sequences.

An alternative embodiment of the invention which also results in the preferred presentation of multiple (e.g. at least 2) copies of the important epitopic regions of the autologous IgE to the immune system is the covalent coupling of the autologous IgE, subsequence or variants thereof to certain molecules. For instance, polymers can be used, e.g. carbohydrates such as dextran, cf. e.g. Lees A et al., 1994, Vaccine 12: 1160-1166; Lees A et al., 1990, J Immunol. 145: 3594-3600, but also mannose and mannan are useful alternatives. Integral membrane proteins from e.g. E. coli and other bacteria are also useful conjugation partners. The traditional carrier molecules such as keyhole limpet hemocyanin (KLH)., tetanus toxoid, diphtheria toxoid, and bovine serum albumin (BSA) are also preferred and useful conjugation partners.

Maintenance of the sometimes advantageous substantial fraction of B-cell epitopes or even the overall tertiary structure of an autologous IgE which is subjected to modification as described herein can be achieved in several ways. One is simply to prepare a polyclonal antiserum directed against the autologous IgE (e.g. an antiserum prepared in a rabbit or another suitable animal) and thereafter use this antiserum as a test reagent (e.g. in a competitive ELISA) against the modified proteins which are produced. Modified versions (analogues) which react to the same extent with the antiserum as does the autologous IgE must be regarded as having the same overall tertiary structure as the autologous IgE whereas analogues exhibiting a limited (but still significant and specific) reactivity with such an antiserum are regarded as having maintained a substantial fraction of the original B-cell epitopes.

Alternatively, a selection of monoclonal antibodies reactive with distinct epitopes on the autologous IgE can be prepared and used as a test panel. This approach has the advantage of allowing 1) an epitope mapping of the autologous IgE and 2) a mapping of the epitopes which are maintained in the analogues prepared.

Of course, a third approach would be resolve the 3-dimensional structure of the autologous IgE or of a biologically active truncate thereof (cf. above) and compare this to the resolved three-dimensional structure of the analogues prepared. Three-dimensional structure can be resolved,by the aid of X-ray diffraction studies and NMR-spectroscopy. Further information relating to the tertiary structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the tertiary structure of a given molecule. However, ultimately X-ray diffraction and/or NMR are necessary to obtain conclusive data since circular dichroism can only provide indirect evidence of correct 3-dimensional structure via information of secondary structure elements.

It should be noted that it is relatively uncomplicated to choose regions in IgE which are specifically suited for introduction of foreign T helper epitopes so as to avoid destructive effects on tertiary structure. Especially preferred regions are flexible loop regions (which do not contribute directly to tertiary structure) as well as flexible hinge regions and N or C termini. Alternatively, the introduction of the T_{H} epitope can be made in a region that has a secondary structure that has a high degree of similarity with the secondary structure of the epitope (an α-helical region may be substituted with an α-helical epitope, a β-sheet region may be substituted with a β-sheet containing epitope etc).

Especially preferred analogues of IgE useful in the present invention are selected from the group consisting of

an amino acid sequence comprising at least two copies of the MIGIS fragment of IgE, wherein at least two MIGIS fragments are separated by at least one foreign T_{H} epitope,

an amino acid sequence comprising a fragment of IgE having an N-terminus in the CH1 or CH2 domain and a C-terminus in the CH4 domain or the MIGIS fragment, wherein at least on foreign T_{H} epitope has been inserted or insubstituted, such as an insubstitution in any one of loops BC, DE, FG, or a loop that faces the CH4 domain,

an amino acid sequence comprising a fragment of IgE having an N-terminus in the CH2 domain and a C-terminus in the CH3 domain, wherein at least one foreign T_{H} epitope has been inserted or in-substituted, such as an insubstitution in any one of loops BC, DE, FG, or a loop that faces the CH4 domain,

an amino acid sequence consisting essentially of a single IgE domain wherein at least one foreign T_{H} epitope has been inserted or in-substituted,

an amino acid sequence comprising at least one of any one of the IgE loop regions and/or at least one of any one of the linker regions, wherein at least one foreign T_{H} epitope separates two IgE derived regions,

an amino acid sequence including the CH3 domain, wherein at least one foreign T_{H} epitope has been introduced so as to substantially destroy a β-sheet stucture in the CH3 domain, and

an amino acid sequence the BC, DE, and FG loops as well as in a loop that faces the CH4 domain. Again, also the resulting expression products of such nucleic acid constructs are embodiments of the present invention,

as well as multimers of any of these that are covalently joined by inert or T_{H} epitope containing linkers. Specific embodiments of such constructs (that are also in their own right parts of the invention) are exemplified in the Examples.

It is important to note that when an IgE construct is prepared by amino acid substitution with a foreign epitope, the introduction is supposed to influence minimally on the epitopes in the relevant IgE fragment. Hence, normally a substitution will only result in an IgE variant where the deleted IgE amino acids constitute 30% or less of the relevant IgE (sub)sequence, and under normal circumstances this number will be much lower such as at most 20%, at most 15%, at most 10%, and at most 7.5%.

It should also be noted that the term “MIGIS fragment” is intended to include not only the MIGIS fragments indicated in the sequence listing herein, but also the various naturally occurring MIGIS fragments that are the results of genetic variation and/or alternative splicing.

In essence there are at present three feasible ways of obtaining the presentation of the relevant epitopes to the immune system: Traditional sub-unit vaccination with polypeptide antigens, administration of a genetically modified live vaccine, and nucleic acid vaccination. These three possibilities will be discussed separately in the following:

Polypeptide Vaccination

This entails administration to the animal in question of an immunogenically effective amount of the at least one first analogue, and, when relevant, administration of an immunologically effective amount of the at least one second analogue. Preferably, the at least one first and/or second analogue(s) is/are formulated together with a pharmaceutically and immunologically acceptable carrier and/or vehicle and, optionally an adjuvant.

When effecting presentation of the analogue to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intradermally, subdermally or intramuscularly. Additional formulations which are suitable for other modes of administration include suppositories and, in some cases, oral, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The analogues may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the polypeptides of the vaccine are sufficiently immunogenic in a vaccine, but for some of the others the immune response will be enhanced if the vaccine further comprises an adjuvant substance. It is especially preferred to use an adjuvant which can be demonstrated to facilitate breaking of the autotolerance to autoantigens.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

Preferred adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the analogues also refer mutatis mutandis to their use in the adjuvant of a vaccine of the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with, bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins such as QuilA and QS21 are interesting. Further possibilities are monophosphoryl lipid A (MPL), and the above mentioned C3 and C3d.

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an analogue of the-present invention can be enhanced by conjugating the antigen to antibodies (or antigen binding antibody fragments) against the Fcγ receptors on monocytes/macrophages. Especially conjugates between analogue and anti-FcγRI have been demonstrated to enhance immunogenicity for the purposes of vaccination.

Other possibilities involve the use of the targeting and immune modulating substances (i.a. cytokines) mentioned above as candidates for the first and second moieties in the modified analogues. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humeral Immune Responses to Small Amounts of Immunogens Using a Novel Medical Device Designated the Virtual Lymph Node”, in: “From the Laboratory to the Clinic, Book of Abstracts, October 12^{th}-15^{th} 1998, Seascape Resort, Aptos, Calif.”.

At any rate, for all (poly)peptide vaccine formulations according to the invention, it is important that, if a CTL response is aimed at, the formulation is capable of shunting the polypeptide immunogen into the MHC type I degradation pathway in-order to ensure that the CTL epitopes of autologous IgE are presented in the context of MHC Class I molecules on the surface of the APC. The skilled person will know which of the above-detailed adjuvants to choose for this specific purpose.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefore the immune system needs to be periodically challenged with the analogues.

Due to genetic variation, different individuals may react with immune responses of varying strength to the same polypeptide. Therefore, the vaccine according to the invention may comprise several different analogues in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different modified or unmodified polypeptides, such as 3-10 different polypeptides. However, normally the number of peptides will be sought kept to a minimum such as 1 or 2 peptides.

Live Vaccines

The second alternative for effecting presentation to the immune system is the use of live vaccine technology. In live vaccination, presentation to the immune 'system is effected by administering, to the animal, a non-pathogenic microorganism which has been transformed with a nucleic acid fragment encoding the necessary epitopic regions or a complete 1^{st} and/or 2^{nd} analogue. Alternatively, the microorganism is transformed with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker P D, 1992, Vaccine 10: 977-990, both incorporated by reference herein. For details about the nucleic acid fragments and vectors used in such live vaccines, cf. the discussion below.

As for the polypeptide vaccine, the T_{H} epitope and/or the first and/or second and/or third moieties can, if present, be in the form of fusion partners to the amino acid sequence derived from the autologous IgE.

As an alternative to bacterial live vaccines, the nucleic acid fragment of the invention discussed below can be incorporated in a non-virulent viral vaccine vector. One possibility is a pox virus such as vaccinia, MVA (modified Vaccinia virus), canary pox, avi-pox, and chicken pox etc. Alternatively, a herpes simplex virus variant can be used.

Normally, the non-pathogenic microorganism or virus is administered only once to the animal, but in certain cases it may be necessary to administer the microorganism more than once in a lifetime.

Also, the microorganism can be transformed with nucleic acid(s) containing regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different open reading frames or at least under the control of different promoters. Thereby it is avoided that the analogue or epitopes are produced as fusion partners to the immunomodulator. Alternatively, two distinct nucleotide fragments can be used as transforming agents.

In order to render a live vaccine highly safe and ensure that it should not be able to trigger degranulation of mast cells and basophils due to cross-linking by anti IgE antibodies of membrane bound IgE, the expression cassette in the live vaccine (especially if it is a virus) can be constructed so as to ensure that no export of the expression product takes place. In this way, only minute amounts of expression product will be exported, whereas the remainder will be processed and presented as peptide fragments in the context of MHC molecules. Hence, no or only a very limited antibody response will be induced, whereas a CTL response will be mounted. This strategy will thus minimize the danger of inducing anaphylaxis.

Nucleic Acid Vaccination

As an alternative to classic administration of a peptide-based vaccine, the technology of nucleic acid vaccination (also known as “nucleic acid immunisation”, “genetic immunisation”, “gene immunisation” and “DNA vaccination) offers a number of attractive features.

First, in contrast to the traditional vaccine approach, nucleic acid vaccination does not require resource consuming large-scale production of the immunogenic agent. (e.g. in the form of industrial scale fermentation of microorganisms producing the analogues necessary in polypeptide vaccination). Furthermore, there is no need to device purification and re-folding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original B-cell epitopes should be preserved in the analogues derived from extracellularly exposed polypeptide sequences, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is best ensured by having the host producing the immunogen.

Two further features render nucleic acid vaccination especially interesting in the context of the present invention. By using DNA as a vaccine agent, it is relatively uncomplicated to ensure presentation of CTL epitopes in the MHC class I context on the APCs. Further, it has been repeatedly demonstrated that immunizations including administration of DNA leads to a shift in T helper cell profile from Th2 to Th1 cells, and since the adverse allergic reactions mediated by IgE are first and foremost supported by Th2 cells, the use of DNA vaccination will in itself provide a beneficial effect on the underlying disease.

Hence, an important embodiment of the method of the invention involves that presentation is effected by in vivo introducing, into the APC, at least one nucleic acid fragment which encodes and expresses the at least one CTL epitope and/or the at least one B-cell epitope, and the at least one first foreign T_{H} epitope (an alternative encompasses administration of at least 2 distinct nucleic acid fragments, where one encodes the at least one CTL epitope and the other encodes the at least one foreign T_{H} epitope). Preferably, this is done by using a nucleic acid fragment which encodes and expresses the above-discussed first analogue. If the first analogue is equipped with the above-detailed T_{H} epitopes and/or first and/or second and/or third moieties, these are then present in the form of fusion partners to the amino acid sequence derived from the autologous IgE, the fusion construct being encoded by the nucleic acid fragment.

As for the traditional vaccination approach, the nucleic acid vaccination can be combined with in vivo introduction, into the APC, of at least one nucleic acid fragment encoding and expressing the second analogue. The considerations pertaining to 1^{st}, 2^{nd} and 3^{rd} moieties and T_{H} epitopes apply also here.

In this embodiment, the introduced nucleic acid is preferably DNA which can be in the form of naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology. The same holds true for other considerations relating to formulation and mode and route of administration and, hence, also these considerations discussed above in connection with a traditional vaccine apply mutatis mutandis to their use in nucleic acid vaccination technology.

One especially preferred type of formulation of nucleic acid vaccines are microparticles containing the DNA. Suitable microparticles are e.g. described in WO 98/31398.

Furthermore, the nucleic acid(s) used as an immunization agent can contain regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different open reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Under normal circumstances, the nucleic acid of the vaccine is introduced in the form of a vector wherein expression is under control of a viral promoter. For more detailed discussions of vectors according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

In order to render a nucleic acid vaccine highly safe and ensure that it should not be able to trigger degranulation of mast cells and basophils due to cross-linking by anti IgE antibodies of membrane bound IgE, the expression cassette in the nucleic acid vaccine can be constructed-so as to ensure that no export of the expression product takes place (e.g. by omitting signal sequences that would result in membrane integration or secretion). In this way, only minute amounts of expression product will be exported, whereas the remainder will be processed and presented as peptide fragments in the context of MHC molecules. Hence, no or only a very limited antibody response will be induced, whereas a CTL response will be mounted. This strategy will thus minimize the danger of inducing anaphylaxis.

An important part of the invention-pertains to a novel method for selecting an appropriate immunogenic analogue of autologous IgE, said immunogenic analogue being capable of inducing a CTL response in the animal against cells displaying an MHC Class I molecule bound to an epitope derived from the autologous IgE. This method comprises the steps of

a) identifying at least one subsequence of the amino acid sequence of autologous IgE, where said subsequence does not contain known or predicted CTL epitopes,

b) preparing at least one putatively immunogenic analogue of the autologous IgE by introducing, in the amino acid sequence of the autologous IgE, at least one T_{H} epitope foreign to the animal in a position within the at least one subsequence identified in step a),

c) and selecting the/those analogues prepared in step b) which are verifiably capable of inducing a CTL response against the autologous IgE in the animal.

Alternatively, the above selection method involves the preparation of a nucleic acid fragment for nucleic acid vaccination purposes. In that situation, it is required that the encoded peptide includes at least one T_{H} epitope.

When the analogue is derived from an part of IgE which is exposed to the extracellular phase, it is preferred that the subsequence identified in step a) further does not contain cysteine residues, or, alternatively, that the T_{H} epitope introduced in step b) does not substantially alter the pattern of cysteine residues. This approach facilitates the preservation of spatial B-cell epitopes in the resulting construct which are similar to the B-cell epitopes in the autologous IgE.

For the same reasons it is preferred that the subsequence identified in step a) further does not contain known or predicted glycosylation sites, or, alternatively, that the T_{H} epitope introduced in step b) does not substantially alter the glycosylation pattern.

Another important consideration pertains to the question of immunological cross-reactivity of the vaccine's polypeptide product with other self-proteins which are not related to a pathology. Such cross-reactivity should preferably be avoided and hence an important embodiment of this method of the invention is one where the subsequence identified in step a) is homologous to an amino acid sequence of a different protein antigen of the animal, and where the introduction of the T_{H} epitope in step b) substantially removes the homology; this means that e.g. regions homologous with other immunoglobulins can be removed so as to avoid adverse effects related to undesired down-regulation of these immunoglobulins.

Related to this embodiment is an embodiment where any amino acid sequences which 1) are not normally exposed to the extracellular phase and 2) which may constitute B-cell epitopes of IgE are not preserved in the analogue. This can be achieved by exchanging such amino acid sequences with T_{H} epitopes which do not constitute B-cell epitopes, by completely removing them, or by partly removing them.

On the other hand, it is preferred that any “true” B-cell epitopes of the autologous IgE are preserved to a high degree, and therefore an important embodiment of the selection method of the invention involves that the introduction in step b) of the foreign T_{H} epitope results in preservation of a substantial fraction of B-cell epitopes of the autologous IgE. It is especially preferred that the analogue preserves the overall tertiary structure of the autologous IgE.

The preparation in step b) is preferably accomplished by molecular biological means or by means of solid or liquid phase peptide synthesis. Shorter peptides are preferably prepared by means of the well-known techniques of solid- or liquid-phase peptide synthesis. However, recent advances in this technology has rendered possible the production of full-length polypeptides and proteins by these means, and-therefore it is also within the scope of the present invention to prepare the long constructs by synthetic means.

After having identified the useful analogues according to the above-discussed method, it is necessary to produce the analogue in larger scale. The polypeptides are prepared according to methods well-known in the art.

This can be done by molecular biological means comprising a first step of preparing a transformed cell by introducing, into a vector, a nucleic acid sequence encoding an analogue which has been selected according to the method and transforming a suitable host cell with the vector. The next step is to culture the transformed cell under conditions facilitating the expression of the nucleic acid fragment encoding the analogue of the autologous IgE, and subsequently recovering the analogue from the culture supernatant or directly from the cells, e.g. in the form of a lysate. Alternatively, the analogue can be prepared by large-scale solid or liquid phase peptide synthesis, cf. above.

Finally, the product can, depending on the cell chosen as a host cell or the synthesis method used, be subjected to artificial post-translational modifications. These can be refolding-schemes known in the art, treatment with enzymes (in order to obtain glycosylation or removal of undesired fusion partners, chemical modifications (again glycosylation is a possibility), and conjugation, e.g. to traditionally carrier molecules.

It should be noted that preferred analogues of the invention (and also the relevant analogues used in the methods of the invention) comprise modifications which results in a polypeptide having a sequence identity of at least 70% with the autologous IgE or with a subsequence thereof of at least 10 amino acids in length. Higher sequence identities are preferred, e.g. at least 75% or even at least 80% or 85%. The sequence identity for proteins and nucleic acids can be calculated as (N_{ref}−N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8)

Nucleic Acid Fragments and Vectors of the Invention

It will be appreciated from the above disclosure that the analogues can be prepared by means of recombinant gene technology but also by means of chemical synthesis or semisynthesis; the latter two options are especially relevant when the modification consists in coupling to protein carriers (such as KLH, diphtheria toxoid, tetanus toxoid, and BSA) and non-proteinaceous molecules such as carbohydrate polymers and of course also when the modification comprises addition of side chains or side groups to an polypeptide-derived peptide chain.

For the purpose of recombinant gene technology, and of course also for the purpose of nucleic acid immunization, nucleic acid fragments encoding the necessary epitopic regions and analogues are important chemical products. Hence, an important part of the invention pertains to a nucleic acid fragment which encodes an analogue described above, preferably a polypeptide wherein has been introduced a foreign T_{H}-cell epitope by means of insertion and/or addition, preferably by means of substitution and/or deletion. The nucleic acid fragments of the invention are either DNA or RNA fragments.

The nucleic acid fragments of the invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector. Preferred cloning and expression vectors of the invention are capable of autonomous replication, thereby enabling high copy-numbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion of or integration into the membrane of the polypeptide fragment, the nucleic acid fragment of the invention, and a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to-be used for effecting in vivo expression in an animal (i.e. when using the vector in DNA vaccination) it is for security reasons preferred that the vector is not capable of being integrated in the host cell genome; typically, naked DNA or non-integrating viral vectors are used, the choices of which are well-known to the person skilled in the art.

The vectors of the invention are used to transform host cells to produce the analogue of the invention. Such transformed cells, which are also part of the invention, can be cultured cells or cell lines used for propagation of the nucleic acid fragments and vectors of the invention, or used for recombinant production of the analogues of the invention. Alternatively, the transformed cells can be suitable live vaccine strains wherein the nucleic acid fragment (one single or multiple copies) have been inserted so as to effect secretion or integration into the bacterial membrane or cell-wall of the analogue.

Preferred transformed cells of the invention are microorganisms such as bacteria (such as the species Escherichia [e.g. E. coli], Bacillus [e.g., Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multi-cellular organism such as a fungus, an insect cell, a plant cell, or a mammalian cell. Most preferred are cells derived from a human being, cf. the discussion of cell lines and vectors below.

For the purposes of cloning and/or optimised expression it is preferred that the transformed cell is capable of replicating the nucleic acid fragment of the invention. Cells expressing the nucleic fragment are preferred useful embodiments of the invention; they can be used for small-scale or large-scale preparation of the analogue or, in the case of non-pathogenic bacteria, as vaccine constituents in a live vaccine.

When producing the analogue of the invention by means of transformed cells, it is convenient, although far from essential, that the expression product is either exported out into the culture medium or carried on the surface of the transformed cell.

When an effective producer cell has been identified it is preferred, on the basis thereof, to establish a stable cell line which carries the vector of the invention and which expresses the nucleic acid fragment encoding the analogue. Preferably, this stable cell line secretes or carries the analogue of the invention, thereby facilitating purification thereof.

In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, dukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available such as Pichia pastoris. For expression in Saccharomyces, the plasmid YRp7, for example, is commonly used (Stinchcomb et al., 1979; Kingsman et al., 1979; Tschemper et al., 1980). This plasmid already contains the trp1 gene which provides a selection marker for a mutant strain of yeast lacking the ability to grow in tryptophan for example ATCC No. 44076 or PEP4-1 (Jones, 1977). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host. cell lines are VERO and HeLa cells, Chinese hamster ovary (CHO) cell lines, and W138, BHK, COS-7 293 and MDCK cell lines.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

Compositions of the Invention

The invention also relates to an immunogenic composition which comprises, as an effective immunogenic agent at least one of the analogues described herein in admixture with a pharmaceutically and immunologically acceptable carrier, vehicle, diluent, or excipient, and optionally an adjuvant, cf. also the discussion of these entities in the description of the method of the invention above.

Furthermore, the invention also relates to a composition for inducing production of antibodies autologous IgE, the composition comprising

a nucleic acid fragment or a vector of the invention, and a pharmaceutically and immunologically acceptable diluent and/or vehicle and/or carrier and/or excipient and/or adjuvant.

Formulation and other specifics concerning such compositions are discussed in the relevant section regarding nucleic acid immunisation above.

In the following examples we present a discussion of the preferred constructs of the invention as well as of their preparation and the testing of the immunological properties of these constructs.

### EXAMPLES

Cloning of the IgE Heavy Chain Gene and Coding Sequences

Plasmids containing the human and murine genes encoding the IgE heavy chain C region and/or the membrane bound IgE heavy chain C region are available from various sources. Also, the sequence information relating to both the human and the murine IgE heavy chain is publicly available.

Isolation or synthesis of genes encoding the CH2-CH3 region will be necessary for constructing the Fc-receptor binding molecule fragments. Isolation or synthesis of genes encoding the membrane bound murine IgE heavy chain part will be necessary for identification of the MIGIS sequence which is disclosed in patents assigned to Tanox Biosystems.

It was originally the intention to isolate the gene fragment encoding entire CH2-CH4 (with and without MIGIS) by the use of either plaque hybridisation and/or PCR technology using conserved primers. However, at a later stage it was decided also to synthesise non-naturally occurring genes encoding the entire CH2-CH3-CH4 (C2-C3-C4) domains and also non-naturally occurring genes encoding the CH2-CH3-CH4-MIGIS (C2-C3-C4-MIGIS)—the genes are non-naturally occurring since they have been codon optimised for expression in mammalian cells and in E. coli, respectively. In general, it is preferred to synthesize the genes because any putative problems with contamination from unknown sources of the IgE encoding material can be overcome and because it is readily possible to optimise codon choices for a relevant expression system.

The sequences are:

DNA encoding C2-C3-C4: SEQ ID NOs: 3 and 5, human IgE, codon choices optimised for mammalian and E. coli expression, respectively, and SEQ ID NOs: 24 and 26, murine IgE, codon choices optimised for mammalian and E. coli expression respectively.

DNA encoding C2-C3-C4-MIGIS: SEQ ID NO: 9, human IgE, codon choices optimised for mammalian expression and SEQ ID NO: 21, murine IgE, codon choices optimised for-mammalian expression.

The artificial and naturally derived constructs thereafter provide the necessary building blocks for a large number of the constructs that are going to be tested according to the present invention. It should be needless to add, that similar constructs that will also include the CH1 domain encoding region can be synthesized in a similar manner—the protein sequences of human IgE C1-C2-C3-C4 and C1-C2-C3-C4-MIGIS are set forth in SEQ ID NOs: 1 and 7, respectively, whereas the corresponding murine sequences are set forth in SEQ ID NOs: 28 and 19, respectively.

Construction of Immunogenic IgE Molecule Fragments

3-D structures derived from human IgE have been determined both in unbound and in FcεRI bound states. This knowledge will be utilized when constructing the linker in the single chain Fc-fragment (scFc) constructs.

A fragment derived from the human IgE heavy chain CH2-CH3 region (301-376, the amino acid numbering corresponding to that of Bennich July 1974, Progress in Immunology II, vol I, pp. 49-58—all numbering of IgE segments that does not explicitly refer to a SEQ ID NO is intended to refer to Bennich's numbering) has been described to compete avidly for binding to the high-affinity IgE receptor (Helm 1988). This fragment has further been used in the construction of a conjugate vaccine and shown that no mast cell stimulatory antibodies were raised. Furthermore, despite the use of these poorly defined and relatively poorly immunogenic constructs, it has anyway been possible to show clearly that the induced autoantibodies could neutralize the pathogenic action of IgE (L. Hellman, 1994). Furthermore, Davis et al. has shown that monoclonal. antibodies directed against the MIGIS region—a short segment derived from the membrane proximal part of the membrane bound IgE heavy chain region—are able to react with B cells expressing membrane bound IgE without interfering with Fc-receptor bound IgE (Davis et al., 1991). Also the CH4 domain is somehow involved in the binding of IgE to the membrane receptor.

It is the intention to construct a variety of immunogenic molecules based on these parts of IgE. The constructs are based on the known human and murine amino acid sequences (cf. SEQ ID NOs: 1, 7, 19, and 28).

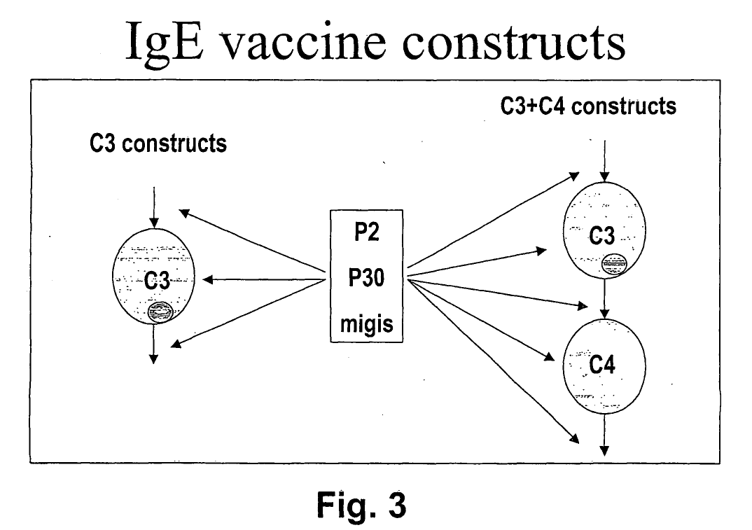

Referring to FIG. 3, the following constructs were initially contemplated where the tetanus toxoid P2 and P30 epitopes are exemplified, but any other T_{H} epitope discussed herein may be utilised:

Construct no. 1 contains several copies of the MIGIS fragment alternating with foreign epitopes, respectively. This type of construct could be very potent at inducing anti-MIGIS antibodies and if formulated correctly, it will be capable of inducing CTLs against B lymphocytes producing IgE. DNA encoding this construct will also in its own right be a potent CTL inducer.

In construct no. 2 a part of the heavy chain mIgE CH2-CH4-Migis fragment has been used (301-547, “Bennich numbering”). In this protein fragment the sequence 377-535 has been substituted with two consecutive copies of the tetanus toxoid P2 and P30 epitopes, respectively. This construct is believed to be able to induce neutralizing antibodies capable of interfering with Fc-receptor binding as well as with B lymphocytes expressing membrane bound IgE.

In construct no. 3 a larger fragment of the CH2-CH4-MIGIS segment has been used (282-547, Bennich numbering). In this fragment 286-300 has subsequently been substituted with one universal T_{H} epitope (P2) and 377-533 has been substituted with another (P30). Based on our previous experiences with inserting T cell epitopes at different positions this fragment may possess different capabilities of inducing neutralizing antibodies compared to construct no. 1 and 2.

In construct no. 4 two copies of construct no. 2 have been linked through an appropriate linker. This is parallel to what have been done previously with antigen binding variable regions from IgG antibodies—the so-called single chain Fv (scFv) fragments—which bind with much higher avidity to the relevant antigen compared to each of the chains alone. We therefore name construct no. 4 a single chain Fc (scFc) fragment and it is likely that such a molecule would be better at mimicking the native relevant IgE Fc part. The linker will be designed based on the known human IgE 3-D structure.

Construct no. 5 is also an scFc fragment. In the murine IgE fragment 301-547 the 381-529 residues have been deleted. Subsequently the remaining fragments have been connected through a linker containing at least two copies of P2 and P30, respectively. In this way P2 and P30 may minimally influence the secondary structure of the relevant parts of the IgE Fc fragment.

Construct no. 6 is also an scFc fragment and consists of two copies of construct no. 2.

Constructs no. 7, 8, and 9 are only based on the 76 amino acid CH2-CH3 sequence of secreted IgE which is involved in Fc receptor binding. In construct no. 7 the foreign epitope has been inserted at position 286-300 of CH2-CH3 282-401 and P30 has been inserted at 377-397. Construct no. 8 is two 301-376 segments connected by the same T cell epitope linker as in construct no. 5. In construct no. 9 P2 is inserted at 377-391 of mIgE segment 301-395. Likewise, P30 has been inserted at positions 377-397 of mIgE 377-401 to create construct no. 10.

Other contemplated constructs also include the CH4 domain. One series of DNA encodes an IgE fragment with the CH2-CH3-CH4 domains wherein has been in-substituted or inserted at least one suitable T_{H} epitope encoding DNA fragment—also the corresponding polypeptide constructs are of course preferred.

One especially preferred construct includes DNA encoding a PADRE epitope (SEQ ID NO: 17) that is inserted, in the human variants, in SEQ ID NO: 3 or 5 after position 12 or, in the murine variants, in SEQ ID NO: 24 or 26 after position 9 (and of course any suitable DNA constructs encoding identical polypeptides where the PADRE peptide is inserted after amino acid 4 in human SEQ ID NO: 1 or after amino acid 3 in murine SEQ ID NO: 23), but the insertion or substitution can be made according to the general AutoVac™ principle, i.e. that if the construct is supposed be able to induce antibodies the introduction of the foreign T_{H} epitope can be made in a region that does not substantially interfere with the majority of the B-cell epitopes of the wild-type IgE, cf. the general description above.

Another preferred group of constructs contains DNA encoding an IgE fragment with the CH2-CH3-CH4-MIGIS domains wherein has been insubstituted or inserted at least one suitable T_{H} epitope encoding DNA fragment—also the corresponding polypeptide construct is of course preferred. One especially preferred construct includes DNA encoding a PADRE epitope (SEQ ID NO: 17) that is inserted in human SEQ ID NO: 9 after position 945 or in murine SEQ ID NO: 21 after position 972 (and of course any suitable DNA constructs encoding identical polypeptides where the PADRE epitope is inserted after amino acid 315 in human SEQ ID NO: 8 or amino acid 324 in murine SEQ ID NO: 20), but the insertion or substitution can be made according to the general AutoVac™ principle, i.e. that if the construct is supposed be able to induce antibodies the introduction of the foreign T_{H} epitope can be made in a region that does not substantially interfere with the majority of the B-cell epitopes of the wild-type IgE, cf. the general description above.

Another group of IgE derived immunogens consists of combinations of the loop regions and/or the linker regions with foreign T-cell help introduced. I.e. such human constructs can be made from DNA encoding the BC loop epitope (SEQ ID NO: 1, positions 244-251) and/or the DE loop epitope (SEQ ID NO: 1, positions 272-280) and/or the FG loop epitope (SEQ ID NO: 1, positions 301-311) and/or the C2C3 linker epitope (SEQ ID NO: 14) and/or the C3C4 linker epitope (SEQ ID NO: 16) in any order or combination with at least one interspersed T-cell epitope. Murine constructs can be made from DNA encoding the BC loop epitope (SEQ ID NO: 34) and/or the DE loop epitope (SEQ ID NO: 32) and/or the FG loop epitope (SEQ ID NO: 30) and/or the C2C3 linker epitope (SEQ ID NO: 36) and/or the C3C4 linker epitope (SEQ ID NO: 38) in any order or combination with at least one interspersed T-cell epitope—both the nucleic acid constructs as well as the protein version of these constructs are part of the present invention. Exemplary constructs include but are not limited to SEQ ID NOs: 11 and 12, as well as constructs having or being encoded by the nucleic acid structure A-P-A and/or A-P-B and/or B-P-B, where A is SEQ ID NO: 13, B is SEQ ID NO: 15 and P is SEQ ID NO: 17, or where A is SEQ ID NO: 13, B is SEQ ID NO: 15 and P is SEQ ID NO: 17.

Yet another class of constructs include insertion of a foreign epitope with the purpose of destroying tertiary structure of the β-sheets of the CH3 domain, i.a. SEQ ID NOs: 1, 2, 7, 8, 19, 20, 23, and 28 where a foreign epitope have been introduced by insertion or substitution in known β-sheet structures in the CH3 part of the sequences—also here, both the nucleic acids encoding such polypeptides as the polypeptides themselves are of course also embodiments of the invention.

Also contemplated are immunogenic constructs based IgE polypeptide such as any one of SEQ ID NOs 1, 2, 7, 8, 19, 20, 23, and 28 where a foreign epitope encoding nucleic acid such as SEQ ID NO: 17 has been introduced in at least one of the BC, DE, and FG loops as well as in a loop that faces the CH4 domain. Again, also the resulting expression products of such nucleic acid constructs are embodiments of the present invention.

Finally, it is also contemplated to prepare nucleic acid constructs where nucleic acids encoding single domains of IgE are “immunogenized” by introduction of foreign T-helper epitopes, such as SEQ ID NO: 17. These and their expression products are also embodiments of the present invention.

Insertion of the T cell epitopes into the truncated IgE molecule is performed by substituting the coding sequence of the expressed part of IgE by traditional molecular biological means using PCR and other conventional molecular biology tools—alternatively, the epitopic sequences are included in completely synthetic genes prepared by conventional DNA synthesis. With regard to the shortest gene fragments the most rational way will certainly be to produce the gene synthetically. This offers a series of advantages since the codon usage can be optimised for the expression system and the mutagenesis will be facilitated by designing the gene with appropriate restriction sites.

Protein Expression and Purification

Purification of IgE: Pure IgE molecules are needed in several of the subsequent assays. Most conveniently these will be purified from sera from allergic patients in a manner known per se. The purified IgE molecules will also be used for production of rabbit antibodies for use in the subsequent analytical work, during purification of the immunogenized constructs, and as a positive control in the functional cell assays.

Expression and purification of the IgE: As soon as the first molecular constructs have been made, the proteins will preferably be expressed in E. coli. Although this will not allow the protein constructs to become glycosylated this organism is preferred due to the relatively low production costs.

A number of the intended IgE constructs are relatively small proteins (app. 12-25 kD) and they will probably all behave very differently during expression, purification and refolding. The procedure will therefore have to be optimised for each construct individually. The purifications will be monitored by SDS-PAGE and Western blotting with polyclonal rabbit anti-IgE antibodies.

We expect to use conventional chromatographic technology during the purification procedures. Probably two ion exchange chromatographic steps in combination with a gel filtration step will be sufficient to obtain >95% pure material.

Screening Procedures

The specificity of anti-IgE antibodies in mice: Groups of mice will be immunized with 25-100 μg each of purified IgE construct either in Freund's adjuvant or in alum, which has previously been used with success. Alum (e.g. Adjuphos) is accepted for human as well as animal use. The mice will probably have to be immunized 3-4 times before they are fully immune. The production of anti-IgE antibodies will be tested using ELISA and native purified IgE as antigen.

The use of mice for selection of IgE molecules will not elucidate whether the molecules eventually also will be immunogenic in dogs. This is, however, very likely based on our previous results obtained with TNFa (Hindersson et al., 1998).

If it is decided to use other animals as an alternative to mice in the selection procedure, groups of 3-5 relevant experimental animals will be immunized with each construct.

The ability of the mouse anti-IgE antibodies to interfere with mast cell degranulation: The mouse anti-IgE sera will be monitored in a relevant mast cell degranulation assay for its ability to reduce e.g. the IgE-induced histamine release from freshly prepared blood basophils or mast cells from allergic subjects. Such assays have already been published.

In order to test the functionality of the constructs containing the MIGIS sequence, it will also be tested whether the sera mentioned above are able to react with B lymphocytes expressing membrane bound IgE. This could be tested using FACS on B cells from allergic subjects if mice sera are used for selection of the constructs. Alternatively the anti-IgE sera could be tested on e.g. L-cells transfected with membrane bound IgE, which also contains the MIGIS sequence.

The ability of the mouse anti-IgE antibodies to inhibit IgE—mediated allergic disorders will be tested in well-established eosinophilia models and in a model comprising transfer of allergen-specific IgE followed by challenge with allergen in mice—mast degranulation

The ability of selected molecules to induce anti-IgE antibodies for Clinical Development: Once 1-3 molecular constructs have been selected based upon the tests mentioned above, larger amounts could be purified for clinical testing.

List of References

Chretien I. et al., J. Immunol. 1988, 141:3128.

Coyle A. J et al., J. Exp. Med. 1996, 183:1303

Dalum I. et al., J. Immunol. 157, 4796-4804, 1996.

Davis F. M. et al., Biotechnology (NY) 1991, 9(1):53.

Fei D. T. et al., J. Immunol. Metods 1994, 171:189.

Helm B. et al., Nature, 1988, 331:180.

Hellman L T., Eur. J. Immunol. 1994, 24:415.

Jardieu P., Curr. Opin. Immunol. 1995, 7 (6):779.

Lustgarten J. et al., Mol. Immunol. 1996, 33:245.

McDonnell J. M. et al., Nature Struct. Biol. 1996, 3:419.

Meng Y. G. et al., Molecular Immunology, 1996, 33 (7/8), 635

Shields R. L. et al., Int. Arch. Allergy Immunol. 1995, 107:308.

1. A method for inducing an immune response against autologous immunoglobulin E (IgE) in an animal, including a human being, the method comprising effecting simultaneous presentation by antigen presenting cells (APCs) of the animal's immune system of an immunogenically effective amount of
at least one CTL epitope derived from the autologous IgE and/or at least one B-cell epitope derived from the autologous IgE, and
at least one first T helper cell epitope (T_{H} epitope) which is foreign to the animal,
wherein the simultaneous presentation is achieved by means of genetic immunisation of the animal.

2. A method for down-regulating autologous IgE in an animal, including a human being by inducing a specific cytotoxic T-lymphocyte (CTL) response against cells producing autologous IgE, the method comprising effecting, in the animal, simultaneous presentation by a suitable antigen presenting cell (APC) of
at least one CTL epitope derived from IgE of the animal, and
at least one first T-helper lymphocyte (T_{H}) epitope which is foreign to the animal,
wherein the simultaneous presentation is achieved by means of genetic immunisation of the animal.

3. The method according to claim 1 or 2, wherein said at least one CTL epitope when presented is associated with an MHC Class I molecule on the surface of the APC and/or wherein said at least one first foreign T_{H} epitope when presented is associated with an MHC Class II molecule on the surface of the APC.

4. The method according to any one of the preceding claims, wherein the APC is a dendritic cell or a macrophage.

5. The method according to any one of the preceding claims, wherein the presentation by the APC of the CTL or B-cell epitope and the first foreign T_{H} epitope is effected by presenting the animal's immune system with at least one first analogue of IgE, said first analogue comprising a variation of the amino acid sequence of IgE, said variation containing at least the CTL epitope and the first foreign T_{H} epitope.

6. The method according to claim 5, wherein the at least first analogue contains a substantial fraction of known and predicted CTL epitopes from the constant domains of the autologous IgE heavy and/or light chain.

7. The method according to claim 6, wherein the substantial fraction of known and predicted CTL epitopes in the amino acid sequence of the analogue are recognized by at least 90% of the MHC-I haplotypes recognizing all known an predicted CTL epitopes in the constant domains of the autologous IgE heavy and/or light chain.

8. The method according to any one of claims 5-7, wherein substantially all known CTL epitopes of the constant domains of the autologous IgE heavy and/or light chain are present in the first analogue and/or wherein substantially all predicted CTL epitopes of the constant domains of the autologous IgE heavy and/or light chain are present in the at least first analogue.

9. The method according to any one of claims 5-8, wherein the at least one first analogue further comprises a part consisting of a modification of the structure of the autologous IgE, said modification having as a result that immunization of the animal with the first analogue induces production of antibodies in the animal against the autologous IgE.

10. The method according to any one of the preceding claims, which further comprises effecting presentation to the animal's immune system of an immunogenically effective amount of at least one second analogue of the autologous IgE, said second analogue containing a modification of the structure of the autologous IgE, said modification having as a result that immunization of the animal with the second analogue induces production of antibodies against the autologous IgE.

11. The method according to claim 10, wherein the modification comprises that at least one second foreign T_{H} epitope is included in the second analogue.

12. The method according to any one of claims 6-11, wherein the first and/or second analogue is/are incapable of inducing an anaphylactic reaction in the animal as a consequence of cross-linking of autologous IgE bound to FcεR-bearing cells by antibodies induced against the first and/or second analogues in the animal.

13. The method according to any one of claims 6-12, wherein the first and/or second analogue(s) comprise(s) a substantial fraction of the B-cell epitopes of the constant domains-of autologous IgE heavy and/or light chain.

14. The method according to any one of claims 6-13, wherein the variation and/or modification involves amino acid substitution and/or deletion and/or insertion and/or addition.

15. The method according to any one of claims 6-14, wherein the variation and/or modification comprises that
at least one first moiety is included in the first and/or second analogue(s), said first moiety effecting targeting of the analogue to an antigen presenting cell (APC), and/or
at least one second moiety is included in the first and/or second analogue(s), said second moiety stimulating the immune system, and/or
at least one third moiety is included in the first and/or second analogue(s), said third moiety optimising presentation of the analogue to the immune system.

16. The method according to any one of claims 5-15, wherein the variation and/or modification includes duplication of at least one B-cell epitope or of at least one CTL epitope of the autologous IgE

17. The method according to any of the preceding claims, wherein the at least one-B-cell epitope is included in or interferes with the FcεR binding region and/or is included in the membrane anchoring region of B-cell bound IgE.

18. The method according to any one of the preceding claims, wherein the first and/or second foreign T_{H} epitope(s) is/are immunodominant and/or wherein the first and/or second foreign T_{H} epitope(s) is/are promiscuous.

19. The method according to any one of claims 5-18, insofar as claims 6-18 are dependent on claim 5, wherein the first and/or second analogue(s) are selected from the group consisting of
an amino acid sequence comprising at least two copies of the MIGIS fragment of IgE, wherein at least two MIGIS fragments are separated by at least one foreign T_{H} epitope,
an amino acid sequence comprising a fragment of IgE having an N-terminus in the CH1 or CH2 domain and a C-terminus in the CH4 domain or the MIGIS fragment, wherein at least on foreign T_{H} epitope has been inserted or insubstituted, such as an insubstitution in any one of loops BC, DE, FG, or a loop that faces the CH4 domain,
an amino acid sequence comprising a fragment of IgE having an N-terminus in the CH2 domain and a C-terminus in the CH3 domain, wherein at least one foreign T_{H} epitope has been inserted or in-substituted, such as an insubstitution in any one of loops BC, DE, FG, or a loop that faces the CH4 domain,
an amino acid sequence consisting essentially of a single IgE domain wherein at least one foreign T_{H} epitope has been inserted or in-substituted,
an amino acid sequence comprising at least one of any one of the IgE loop regions and/or at least one of any one of the linker regions, wherein at least one foreign T_{H} epitope separates two IgE derived regions,
an amino acid sequence including the CH3 domain, wherein at least one foreign T_{H} epitope has been introduced so as to substantially destroy a β-sheet stucture in the. CH3 domain, and
an amino acid sequence the BC, DE, and FG loops as well as in a loop that faces the CH4 domain.

20. The method according to any one of claims 11-19, wherein the first and/or second foreign T_{H} epitope(s) is/are selected from a natural T_{H} epitope and an artificial MHC-II binding peptide sequence.

21. The method according to claim 20, wherein the natural T_{H} epitope is selected from a Tetanus toxoid epitope such as P2 or P30, a diphtheria toxoid epitope, an influenza virus hemagluttinin epitope, and a P. falciparum CS epitope.

22. The method according to any one of claims 11-21, wherein the first and/or second T_{H} epitopes and/or first and/or second and/or third moieties are present in the form of fusion partners to the amino acid sequence derived from the autologous IgE.

23. The method according to claim 22, wherein the first moiety is a substantially specific binding partner for an APC specific surface antigen or wherein the first moiety is a hapten.

24. The method according to any one of claims 15-23, wherein the second moiety is a cytokine selected from interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13. (IL-13), interleukin 15 (IL-15), and granulocyte-macrophage colony stimulating factor (GM-CSF), or an effective part thereof; a heat-shock protein selected from HSP70, HSP90, HSC70, GRP94, and calreticulin (CRT), or an effective part thereof; or a hormone.

25. The method according to any one of claims 15-24, wherein the third moiety is a lipid such as a palmitoyl group, a myristyl group, a farnesyl group, a geranyl-geranyl group, a GPI-anchor, and an N-acyl diglyceride group.

26. The method according to claim any one of claims 5-25, wherein the first and/or second analogue(s) has/have substantially the overall tertiary structure of the constant domains of autologous IgE heavy and/or light chain.

27. The method according to any one of claims 5-26, wherein is also administered, to the animal, an immunogenically effective amount of the at least one first analogue.

28. The method according to claim 27, wherein is also administered an immunologically effective amount of the at least one second analogue.

29. The method according to claim 27 or 28, wherein said at least one first and/or second analogue(s) is/are formulated together with a pharmaceutically and immunologically acceptable carrier and/or vehicle and, optionally an adjuvant.

30. The method according to claim 29, wherein said adjuvant facilitates uptake by APCs, such as dendritic cells, of the at least first and/or second analogues.

31. The method according to claim 30, wherein the adjuvant is selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant.

32. The method according to claim 31, wherein the cytokine is as defined as in claim 24, or an effective part thereof, wherein the toxin is selected from the group consisting of listeriolycin (LLO), Lipid A (MPL, L180.5/RalLPS), and heat-labile enterotoxin, wherein the mycobacterial derivative is selected from the group consisting of muramyl dipeptide, complete Freund's adjuvant, RIBI, and a diester of trehalose such as TDM and TDE, wherein the immune targeting adjuvant is selected from the group consisting of CD40 ligand, CD40 antibodies or specifically binding fragments thereof, mannose, a Fab fragment, and CTLA-4, wherein the oil formulation comprises squalene or incomplete Freund's adjuvant, wherein the polymer is selected from the group consisting of a carbohydrate such as dextran, PEG, starch, mannan, and mannose; a plastic polymer such as; and latex such as latex beads, wherein the saponin is Quillaja saponaria saponin, Quil A, and QS21, and wherein the particle comprises latex or dextran.

33. The method according to any one of claims 27-32, which includes administration via a route selected from the oral route and the parenteral route such as the intradermal, the subdermal, the peritoneal, the buccal, the sublingual, the epidural, the spinal, the anal, and the intracranial routes.

34. The method according to any of claim 27-33, which includes at least one administration a year, such as at least 2, 3, 4, 5, 6, and 12 administrations a year.

35. The method according to any one of claims 1-4, wherein presentation is effected by in vivo introducing, into the APC, at least one nucleic acid fragment which encodes and expresses the at least one CTL epitope and/or the at least one B-cell epitope, and the at least one first foreign T_{H} epitope.

36. The method according to any one of claims 5-14, wherein presentation is effected by in vivo introducing, into the APC, at least one nucleic acid fragment encoding and expressing the first analogue.

37. The method according to any one of claims 15-26, wherein the T_{H} epitope and/or the first and/or second and/or third moieties are present in the form of fusion partners to the amino acid sequence derived from the autologous IgE, and wherein presentation is, effected by in vivo introducing, into the APC, at least one nucleic acid fragment encoding and expressing the first and/or second analogue.

38. The method according to any one of claims 11-14 and 36, which further comprises in vivo introduction, into the APC, of at least one nucleic acid fragment encoding and expressing the second analogue.

39. The method according to any one of claims 1-4, wherein presentation is effected by in viva co-introducing, into the APC, at least two nucleic acid fragments, wherein one encodes and expresses the at least one CTL epitope and wherein another encodes and expresses the at least one first foreign T_{H} epitope, and wherein the first foreign T_{H} epitope is as defined in any one of claims 1, 2 and 21-24.

40. The method according to any one of claims 35-39, wherein the nucleic acid fragment~s) introduced is/are selected from naked DNA, DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with a targeting carbohydrate, DNA formulated with Calcium precipitating agents, DNA coupled to an inert carrier molecule, and DNA formulated with an adjuvant.

41. The method according to claim 40, wherein the adjuvant is selected from the group consisting of the adjuvants defined in any one of claims 30-32.

42. The method according to any one of claims 35-41, wherein the mode of administration is as defined in claim 33 or 34.

43. A composition for inducing production of antibodies against IgE, the composition comprising
a nucleic acid fragment encoding a first analogue as defined in any one of claims 5-9 and 12-26, and
a pharmaceutically and immunologically acceptable diluent and/or vehicle and/or adjuvant.


SEQUENCE LISTING (38 sequences)

SEQ ID NO: 1 - protein, 428 aa, Homo sapiens
  DOMAIN (11)..(116): Human IgE heavy chain C1 domain
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 2 - protein, 323 aa, Homo sapiens
  DOMAIN (8)..(103): Human IgE heavy chain C2 domain
MRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - DNA, 975 bp, Artificial Sequence
  CDS (1)..(975): Artificial DNA sequence codon optimized for expression in mammalian cells of human IgE heavy chain fragment spanning C2, C3, and C4.
atgcgggactttactcctccaaccgtgaaaattcttcagagcagctgcgatggagggggacacttcccccctacaattcagctcctgtgtctggtcagtggttacacaccaggcactatcaatatcacctggctggaagatggccaggtgatggacgtagacctctccaccgcctctactacgcaggaaggcgaactcgcaagtactcagtcagagctcaccctgtcccaaaagcattggttgtcagatcgaacctatacatgccaggttacttatcagggccataccttcgaagacagcacaaaaaagtgtgctgactcaaatcccagaggggtcagcgcctacctgagcagaccttctcccttcgacctgtttatcaggaaatcccctacgatcacttgtcttgtggtcgatcttgccccatctaagggcacagtcaacctgacctggagtcgggcctccggaaagccagttaatcattcaacccggaaggaagagaaacagaggaatggcaccctcaccgttaccagcacactgcctgtgggcactagagactggatagaaggagagacttaccagtgtcgcgtcacacatccacacctgccgcgagcattgatgagatccaccacaaagacgagtggtccgcgggctgctcctgaggtttatgcattcgcaacccccgagtggcctgggtcccgagataagagaacactcgcttgcttgatccaaaactttatgccggaggatatttccgtgcagtggctgcacaacgaggtgcagctccctgatgcccgccactctactacccaaccccgcaaaacaaaggggagcgggtttttcgtattctcccggcttgaggtgacacgcgcggagtgggagcaaaaggacgaatttatttgcagggccgtgcacgaagctgcgtccccctctcagacggtacagagggccgtgtctgtgaaccccggcaaatgataa

SEQ ID NO: 4 - protein, 323 aa, Artificial Sequence; identical to SEQ ID NO: 2
  Artificial protein sequence optimized for expression in mammalian cells of human IgE heavy chain fragment spanning C2, C3, and C4.

SEQ ID NO: 5 - DNA, 972 bp, Artificial Sequence
  CDS (1)..(972): Artificial DNA sequence codon optimized for expression in E. coli of human IgE heavy chain fragment spanning C2, C3, and C4
atgcgtgacttcacgccgccgactgtcaaaatcctgcagtccagttgcgacggtggcggtcatttcccgccgaccatccagctgctgtgcctggttagcggttatacccctggcaccatcaatatcacctggctggaagacggtcaggttatggatgtcgacctgtctaccgcctctaccacccaggaaggtgaactggcttctacccagtctgaactgaccctgtctcagaaacactggctgtctgaccgtacctacacctgtcaggttacctatcagggtcacaccttcgaagattctaccaagaaatgcgctgactccaatccgcgtggcgtttctgcttacctgtctcgtccgtctccctttgatctgttcattcgtaaaagcccgaccattacctgcctggttgttgacctggcaccaagcaaaggtaccgttaacctgacctggtctcgtgcaagcggtaaaccggttaaccactctacgcgtaaagaagagaagcaacgtaacggcaccctgacggttacctctaccctgccggttggtacccgtgactggatcgaaggtgaaacctaccagtgccgcgttacccacccgcatctgccgcgcgctctgatgcgttcgaccaccaaaacctctggtccgcgtgctgctccggaagtttacgctttcgctaccccggaatggccgggctctcgtgacaaacgtaccctggcttgcctgatccagaacttcatgccggaagatatttccgttcagtggctgcacaatgaagttcaactgccggacgctcgccatagtacaacccagccgcgtaaaacgaaaggttctggcttttttgtattcagccgtctggaagttacccgtgctgaatgggaacagaaagacgaatttatctgccgcgctgttcacgaagccgctagtccgtctcagaccgttcagcgtgctgtttctgttaacccgggtaaataa

SEQ ID NO: 6 - protein, 323 aa, Artificial Sequence; identical to SEQ ID NO: 2
  Artificial protein sequence optimized for expression in E. coli of human IgE heavy chain fragment spanning C2, C3, and C4

SEQ ID NO: 7 - protein, 441 aa, Homo sapiens
  DOMAIN (11)..(106): IgE heavy chain C1 domain
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPELDVCVEEAEGEAPW

SEQ ID NO: 8 - protein, 336 aa, Homo sapiens
  DOMAIN (8)..(103): IgE heavy chain C2 domain
MRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPELDVCVEEAEGEAPW

SEQ ID NO: 9 - DNA, 996 bp, Artificial Sequence
  CDS (1)..(996): Artificial DNA sequence with codons optimised for expression in mammalian cells of human IgE fragment spanning C2, C3, C4 and MIGIS.
atgcgggactttactcctccaaccgtgaaaattcttcagagcagctgcgatggagggggacacttcccccctacaattcagctcctgtgtctggtcagtggttacacaccaggcactatcaatatcacctggctggaagatggccaggtgatggacgtagacctctccaccgcctctactacgcaggaaggcgaactcgcaagtactcagtcagagctcaccctgtcccaaaagcattggttgtcagatcgaacctatacatgccaggttacttatcagggccataccttcgaagacagcacaaaaaagtgtgctgactcaaatcccagaggggtcagcgcctacctgagcagaccttctcccttcgacctgtttatcaggaaatcccctacgatcacttgtcttgtggtcgatcttgccccatctaagggcacagtcaacctgacctggagtcgggcctccggaaagccagttaatcattcaacccggaaggaagagaaacagaggaatggcaccctcaccgttaccagcacactgcctgtgggcactagagactggatagaaggagagacttaccagtgtcgcgtcacacatccacacctgccgcgagcattgatgagatccaccacaaagacgagtggtccgcgggctgctcctgaggtttatgcattcgcaacccccgagtggcctgggtcccgagataagagaacactcgcttgcttgatccaaaactttatgccggaggatatttccgtgcagtggctgcacaacgaggtgcagctccctgatgcccgccactctactacccaaccccgcaaaacaaaggggagcgggtttttcgtattctcccggcttgaggtgacacgcgcggagtgggagcaaaaggacgaatttatttgcagggccgtgcacgaagctgcgtccccctctcagacggtacagagggagctggacgtgtgcgtggaggaggccgagggcgaggccccctggtgataa

SEQ ID NO: 10 - protein, 330 aa, Artificial Sequence
  Artificial protein sequence optimised for expression in mammalian cells of human IgE fragment spanning C2, C3, C4 and MIGIS.
MRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRELDVCVEEAEGEAPW

SEQ ID NO: 11 - DNA, 171 bp, Artificial Sequence
  CDS (1)..(165): Synthetic DNA sequence encoding artificial sequence
atggtcacacatccacacctgccgcgagcattgatggctaagttcgtggccgcttggaccctgaaggccgcagctcttgccccatctaagggcacagtcgctaagttcgtggccgcttggaccctgaaggccgcagctaaacagaggaatggcaccctcaccgtttgataa

SEQ ID NO: 12 - protein, 55 aa, Artificial Sequence
MVTHPHLPRALMAKFVAAWTLKAAALAPSKGTVAKFVAAWTLKAAAKQRNGTLTV

SEQ ID NO: 13 - DNA, 45 bp, Artificial Sequence
  CDS (1)..(42): Synthetic DNA sequence encoding linker between human IgE domains C2 and C3
agcacaaaaaagtgtgctgactcaaatcccagaggggtcagcgcc

SEQ ID NO: 14 - protein, 14 aa, Artificial Sequence
  Protein sequence of C2C4 linker epitope directed to Homo sapiens
STKKCADSNPRGVS

SEQ ID NO: 15 - DNA, 27 bp, Artificial Sequence
  CDS (1)..(27): Synthetic DNA sequence encoding linker between human IgE domains C3 and C4
acaaagacgagtggtccgcgggctgct

SEQ ID NO: 16 - protein, 9 aa, Artificial Sequence
  Protein sequence of C3C4 linker epitope directed to Homo sapiens
TKTSGPRAA

SEQ ID NO: 17 - DNA, 39 bp, Artificial Sequence
  CDS (1)..(39): Synthetic DNA sequence encoding pan DR binding epitope
gctaagttcgtggccgcttggaccctgaaggccgcagct

SEQ ID NO: 18 - protein, 13 aa, Artificial Sequence
  A preferred T-cell epitope having a lack of MHC
AKFVAAWTLKAAA

SEQ ID NO: 19 - protein, 432 aa, mus musculus
  MISC_FEATURE (1)..(432): Murine IgE heavy chain, domains C1, C2, C3, C4, and MIGIS fragment
SIRNPQLYPLKPCKGTASMTLGCLVKDYFPNPVTVTWYSDSLNMSTVNFPALGSELKVTTSQVTSWGKSAKNFTCHVTHPPSFNESRTILVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLELDLQDLCIEEVEGEELEE

SEQ ID NO: 20 - protein, 343 aa, mus musculus
  MISC_FEATURE: Murine IgE heavy chain, domains C2, C3, C4, and MIGIS fragment
MVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLELDLQDLCIEEVEGEELEE

SEQ ID NO: 21 - DNA, 1035 bp, Artificial Sequence
  CDS (1)..(1035): Artificial DNA sequence codon optimised for mammalian expression of murine IgE heavy chain fragment including domains C2, C3, C4, and MIGIS.
atggtgagacccgtgaacattaccgaacctacactggagctgctccattcctcttgtgatcctaacgctttccatagcaccattcagctctactgttttatctatggccacatcctgaacgatgtgtctgtcagctggctgatggatgaccgcgagatcaccgataccctcgctcagactgtcctgatcaaagaagagggcaaactcgcctctacttgttccaaactgaacatcaccgagcagcagtggatgtccgaaagcacattcacgtgcaaggtgacgagccagggcgtggactatctggcccacaccaggcggtgccccgaccacgaaccccgaggcgtgattacttacctgatccctccctcccctctggacctgtaccagaacggcgctcctaagctgacttgcctggtggtggacctggagtctgagaagaatgtcaatgtcacatggaatcaggagaagaagacctccgtgtctgcctctcagtggtacacaaagcaccacaataacgctaccacctccatcacatctattctgccagtggtcgctaaggactggatcgagggctatggctatcagtgcatcgtcgaccacccagacttccccaagcctattgtcagatctatcacaaagacccctggccagagaagcgctcccgaggtgtacgtgttcccccctccagaggaggagagcgaggataagagaaccctgacatgtctgatccagaatttttttcccgaagatatttccgtgcagtggctgggcgatggcaagctgattagcaatagccagcatagcacaacaacaccactgaaatccaacggctctaaccagggctttttcattttcagcagactggaagtggccaaaaccctgtggacccagagaaaacagttcacatgccaggtgatccatgaggccctccagaaaccaagaaagctggaaaagacaatctccacctccctggagctggacctccaggacctgtgcatcgaagaggtggaaggcgaggaactggaagagtaatga

SEQ ID NO: 22 - protein, 343 aa, Artificial Sequence; identical to SEQ ID NO: 20
  Artificial protein sequence optimised for mammalian expression of murine IgE heavy chain fragment including domains C2, C3, C4, and MIGIS.

SEQ ID NO: 23 - protein, 332 aa, mus musculus
  MISC_FEATURE (1)..(332): Murine IgE heavy chain domains C2, C3, and C4.
MVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 24 - DNA, 999 bp, Artificial Sequence
  CDS (1)..(999): Artificial DNA sequence codon optimised for expression in mammali an cells of murine IgE fragment including C2, C3, and C4.
atggtgagacccgtgaacattaccgaacctacactggagctgctccattcctcttgtgatcctaacgctttccatagcaccattcagctctactgttttatctatggccacatcctgaacgatgtgtctgtcagctggctgatggatgaccgcgagatcaccgataccctcgctcagactgtcctgatcaaagaagagggcaaactcgcctctacttgttccaaactgaacatcaccgagcagcagtggatgtccgaaagcacattcacgtgcaaggtgacgagccagggcgtggactatctggcccacaccaggcggtgccccgaccacgaaccccgaggcgtgattacttacctgatccctccctcccctctggacctgtaccagaacggcgctcctaagctgacttgcctggtggtggacctggagtctgagaagaatgtcaatgtcacatggaatcaggagaagaagacctccgtgtctgcctctcagtggtacacaaagcaccacaataacgctaccacctccatcacatctattctgccagtggtcgctaaggactggatcgagggctatggctatcagtgcatcgtcgaccacccagacttccccaagcctattgtcagatctatcacaaagacccctggccagagaagcgctcccgaggtgtacgtgttcccccctccagaggaggagagcgaggataagagaaccctgacatgtctgatccagaatttttttcccgaagatatttccgtgcagtggctgggcgatggcaagctgattagcaatagccagcatagcacaacaacaccactgaaatccaacggctctaaccagggctttttcattttcagcagactggaagtggccaaaaccctgtggacccagagaaaacagttcacatgccaggtgatccatgaggccctccagaaaccaagaaagctggaaaagacaatctccacctccctgggcaacaccagcctgagaccttcttaa

SEQ ID NO: 25 - protein, 332 aa, Artificial Sequence; identical to SEQ ID NO: 23
  Artificial protein sequence optimized for expression in mammalian cells of murine IgE heavy chain fragment spanning C2, C3, and C4.

SEQ ID NO: 26 - DNA, 999 bp, Artificial Sequence
  CDS (1)..(999): Artificial DNA sequence codon optimized for expression in E. coli of IgE heavy chain domains C2, C3, and C4
atggttcgtccggtgaacatcaccgaaccaacgctggaattgctgcatagctcctgcgatccgaatgcttttcacagtaccattcagttatattgttttatctacggccatattctgaacgatgtcagcgtttcttggcttatggacgatcgcgagatcactgataccctggcccaaacagtgctgattaaagaagaaggtaagctcgcatcaacctgctcgaaactgaatatcactgagcagcagtggatgagcgaatccaccttcacgtgtaaagtcactagtcaaggcgttgactatttggcgcacacccgtcgctgccctgatcatgagccgcgtggggtcattacctacctgatcccaccgtcgcctttagatctgtatcagaacggagcgccgaagctcacatgtctggtggtagaccttgaatcagagaaaaatgttaacgtgacctggaatcaggaaaagaaaacgtccgtcagcgctagtcaatggtataccaagcatcacaacaatgcaacgactagcattacctccatcctgcctgttgtggccaaagattggattgaaggctacggttatcagtgtatcgtagatcatccggactttccaaaaccgattgttcgctcgatcacgaagaccccagggcagcgttccgctcctgaagtctacgtgtttccgcctccagaggaagaaagtgaggataaacgcacattaacctgcctgattcaaaacttctttcccgaagatatcagcgttcagtggttgggcgacggtaaactgatttccaattcacagcacagcacgactaccccgcttaagagtaacggctccaatcaaggttttttcatcttttcgcgtctggaagtggcgaaaaccctctggacgcagcgcaaacagtttacctgtcaagtcattcatgaggcactgcaaaagcctcgtaaactggaaaagacaatcagtaccagcttaggaaacacgtccctgcgcccgtcgtaa

SEQ ID NO: 27 - protein, 332 aa, Artificial Sequence; identical to SEQ ID NO: 23
  Artificial protein sequence optimized for expression in mammalian cells of murine IgE heavy chain fragment spanning C2, C3, and C4.

SEQ ID NO: 28 - protein, 421 aa, mus musculus
  MISC_FEATURE: Murine IgE heavy chain domains C1, C2, C3, and C4.
SIRNPQLYPLKPCKGTASMTLGCLVKDYFPNPVTVTWYSDSLNMSTVNFPALGSELKVTTSQVTSWGKSAKNFTCHVTHPPSFNESRTILVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 29 - DNA, 33 bp, Artificial Sequence
  CDS (1)..(33): Artificial DNA sequence encoding epitope in the FG loop of murine IgE heavy chain.
gtcgaccacccagacttccccaagcctattgtc

SEQ ID NO: 30 - protein, 11 aa, Artificial Sequence
  Artificial protein sequence encoding epitope in the FG loop of murine IgE heavy chain.
VDHPDFPKPIV

SEQ ID NO: 31 - DNA, 36 bp, Artificial Sequence
  CDS (1)..(36): Artificial DNA sequence encoding epitope in DE loop of murine IgE.
aagcaccacaataacgctaccacctccatcacatct

SEQ ID NO: 32 - protein, 12 aa, Artificial Sequence
  Artificial protein sequence encoding epitope in DE loop of murine IgE.
KHHNNATTSITS

SEQ ID NO: 33 - DNA, 27 bp, Artificial Sequence
  CDS (1)..(27): Artificial DNA sequence encoding epitope in BC loop of murine IgE heavy chain.
ctggagtctgagaagaatgtcaatgtc

SEQ ID NO: 34 - protein, 9 aa, Artificial Sequence
  Artificial protein sequence encoding epitope in BC loop of murine IgE heavy chain.
LESEKNVNV

SEQ ID NO: 35 - DNA, 45 bp, Artificial Sequence
  CDS (1)..(45): Artificial DNA sequence encoding epitope including linker between the murine IgE heavy chain C2 and C3 domains.
gcccacaccaggcggtgccccgaccacgaaccccgaggcgtgatt

SEQ ID NO: 36 - protein, 15 aa, Artificial Sequence
  Artificial protein sequence encoding epitope including linker between the murine IgE heavy chain C2 and C3 domains.
AHTRRCPDHEPRGVI

SEQ ID NO: 37 - DNA, 27 bp, Artificial Sequence
  CDS (1)..(27): Artificial DNA sequence encoding epitope including linker between the murine IgE heavy chain C3 and C4 domains.
acaaagacccctggccagagaagcgct

SEQ ID NO: 38 - protein, 9 aa, Artificial Sequence
  Artificial protein sequence encoding epitope including linker between the murine IgE heavy chain C3 and C4 domains.
TKTPGQRSA